(19) United States
(12) Patent Application Publication
(10) Pub. No.: US 2017/0224431 A1
(43) Pub. Date: Aug. 10, 2017

(54) Medical Device Case

(71) Applicant: beteSTRONGCASES, LLC, Northfield, IL (US)

(72) Inventors: Connor F. Mitchell, Skokie, IL (US); Patrick M. Harris, Downers Grove, IL (US)

(73) Assignee: beteSTRONGCASES, LLC

(21) Appl. No.: 15/494,651

(22) Filed: Apr. 24, 2017

Related U.S. Application Data
(63) Continuation-in-part of application No. 14/704,637, filed on May 5, 2015, now Pat. No. 9,629,954.

(51) Int. Cl. A61B 50/31; A61M 5/142; B65D 43/22; E05C 19/14; E05B 65/52; B65D 43/16; A61J 1/16; F16L 5/02

(52) U.S. Cl. CPC A61B 50/31; A61J 1/16; A61M 5/14244; F16L 5/02; E05C 19/14; E05B 65/5276; B65D 43/16; B65D 43/22; A61M 2205/507; A61B 2050/3008

(57) ABSTRACT

A medical device case for allowing passage of a tube from the interior to the exterior of a case such that the case is sealed against fluid intrusion whether a tube is present or absent. The medical device case generally includes a case having a first portion and a second portion hingedly connected to the first portion. The first portion and second portion may be closed together to define a compartment in which a medical device may be positioned. A tube seal assembly is included which will allow a tube leading from the medical device within the compartment to exit the case through a passage. The tube seal assembly generally includes a first seal and second seal which combine when the case is closed to seal around the tube when it is present or to seal against each other when no tube is present.

### CROSS REFERENCE TO RELATED APPLICATIONS

I hereby claim benefit under Title 35, United States Code, Section 120 of U.S. patent application Ser. No. 14/704,637 filed May 5, 2015. This application is a continuation-in-part of the Ser. No. 14/704,637 application. The Ser. No. 14/704,637 application is currently pending. The Ser. No. 14/704,637 application is hereby incorporated by reference into this application.

### STATEMENT REGARDING FEDERALLY SPONSORED RESEARCH OR DEVELOPMENT

Not applicable to this application.

### BACKGROUND

Field

Example embodiments in general relate to a Medical Device Case for allowing passage of a tube from the interior to the exterior of a case such that the case is sealed against leakage whether a tube is present or absent.

Related Art

Any discussion of the related art throughout the specification should in no way be considered as an admission that such related art is widely known or forms part of common general knowledge in the field.

It is often desirable to store or position a medical device within a case. The case will prevent the fluids within the device from spilling and potentially contaminating an area or creating hazardous conditions in the case of device failure or damage. In a typical prior art case, the sealing element is a free gasket or seal which is clamped between the two case halves. In some prior art, the free gasket or seal is instead bonded to one of the case halves.

When sealing against a round tube, this one-element seal prior art configuration does not function due to the inability of the hard case section to deform enough to seal against the tube. Where two matching and opposing sealing elements are utilized to provide a channel for the tube to pass through, leakage may occur when the tube is not present between the sealing elements and thus create the risk of exposure if the medical device within the case becomes ruptured or otherwise compromised or ingress through the tube passage if the exterior of the case is immersed in or exposed to liquid.

Because of the inherent problems with the related art, there is a need for a new and improved tube passage sealing system for allowing passage of a tube from the interior to the exterior of a case such that the case is sealed against leakage whether a tube is present or absent.

### SUMMARY

An example embodiment of the present invention is directed to a medical device case. The medical device case includes a case having a first portion and a second portion hingedly connected to the first portion. The first portion and second portion may be closed together to define a compartment in which a medical device may be positioned. A tube seal assembly is included which will allow a tube leading from the medical device within the compartment to exit the case through a passage. The tube seal assembly generally includes a first seal and second seal which combine when the case is closed to seal around the tube when it is present or to seal against each other when no tube is present.

There has thus been outlined, rather broadly, some of the features of the medical device case in order that the detailed description thereof may be better understood, and in order that the present contribution to the art may be better appreciated. There are additional features of the medical device case that will be described hereinafter and that will form the subject matter of the claims appended hereto. In this respect, before explaining at least one embodiment of the medical device case in detail, it is to be understood that the medical device case is not limited in its application to the details of construction or to the arrangements of the components set forth in the following description or illustrated in the drawings. The medical device case is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

Example embodiments will become more fully understood from the detailed description given herein below and the accompanying drawings, wherein like elements are represented by like reference characters, which are given by way of illustration only and thus are not limitative of the example embodiments herein.

FIG. 1 is an upper perspective view of the present invention with the case closed.

FIG. 2 is an upper perspective view of the present invention with the case opened and a medical device aligned for insertion with the case.

FIG. 3 is an upper perspective view of the medical device positioned within the case of the present invention.

FIG. 4 is an upper perspective view illustrating a tube extending through the passage of the case of the present invention.

FIG. 5 is a top view of the case of the present invention in a closed position without a medical device positioned therein.

FIG. 6 is a top view of an embodiment of the case which includes a pair of tube seals to accommodate a pair of tubes.

FIG. 7 is a frontal view of the case of the present invention in a closed position without a medical device positioned therein.

FIG. 8 is a sectional view taken along line 8-8 of FIG. 7.

FIG. 9 is a side sectional view of the present invention with a medical device positioned within the container.

FIG. 10a is a frontal view illustrating the deformation of the tube seals around a tube of the present invention to seal the case when a tube is present taken along line 10-10 of FIG. 9.

FIG. 10b is a close-up frontal view illustrating the deformation of the tube seals around a tube of the present invention to seal the case when a tube is present taken along line 10-10 of FIG. 9.

FIG. 11 is a close-up upper perspective view of a first embodiment of a tube seal of the present invention.

FIG. 12 is a close-up upper perspective view of a second embodiment of a tube seal of the present invention.

FIG. 13 is a close-up upper perspective view of a third embodiment of a tube seal of the present invention.

FIG. 14 is a close-up upper perspective view of a third embodiment of a tube seal of the present invention with ribs.

FIG. 15 is a close-up upper perspective of the third embodiment of the tube seal of the present invention with a second embodiment of ribs.

FIG. 16 is a side upper perspective view of the present invention with a displacement opening in the case.

FIG. 17 is a sectional view taken along line 17-17 of FIG. 16.

FIG. 18 is a sectional view taken along line 18-18 of FIG. 17.

FIG. 19 is an upper perspective view of an alternate embodiment of the present invention in which a perimeter engagement member is used on the second portion of the case.

FIG. 20 is a top cutaway view of a third embodiment of the tube seal of the present invention.

FIG. 21 is a top cutaway view of a third embodiment of the tube seal of the present invention with ribs.

FIG. 22 is an upper perspective view of an alternate embodiment of the present invention in a closed state.

FIG. 23 is an upper perspective view of an alternate embodiment of the present invention with its clasp opened.

FIG. 24 is an upper perspective view of an alternate embodiment of the present invention in an opened state with a device installed.

FIG. 25 is a first side upper perspective view of a medical device case in accordance with an example embodiment.

FIG. 26 is a second side upper perspective view of a medical device case in accordance with an example embodiment.

FIG. 27 is an upper perspective view of a medical device case in a closed state with a tube exiting the case in accordance with an example embodiment.

FIG. 28 is an upper perspective view of a medical device case in a closed state with the clasp unlatched in accordance with an example embodiment.

FIG. 29 is an upper perspective view of a medical device case in an opened state in accordance with an example embodiment.

FIG. 30 is an upper perspective view of a medical device case in an opened state with a first block spacer installed in accordance with an example embodiment.

FIG. 31 is an upper perspective view of a medical device case in an opened state with a first medical device positioned in the case in accordance with an example embodiment.

FIG. 32 is an upper perspective view of a medical device case in an opened state with a block spacer installed and a medical device aligned for positioning within the case in accordance with an example embodiment.

FIG. 33 is an upper perspective view of a medical device case in an opened state with a block spacer and medical device aligned for positioning within the case in accordance with an example embodiment.

FIG. 34 is an upper perspective view of a medical device case in an opened state with a block spacer and medical device positioned within the case in accordance with an example embodiment.

FIG. 35 is an upper perspective view of a medical device case in an opened state with a second block spacer positioned within the case in accordance with an example embodiment.

FIG. 36 is an upper perspective view of a medical device case in an opened state with a block spacer and medical device aligned for positioning within the case in accordance with an example embodiment.

FIG. 37 is a top view of a medical device case in an opened state in accordance with an example embodiment.

FIG. 38a is a side sectional view of a medical device case in a closed state with the clasp fully engaged in accordance with an example embodiment.

FIG. 38b is a side sectional view of a medical device case in a closed state illustrating disengagement of the latch lever in accordance with an example embodiment.

FIG. 38c is a side sectional view of a medical device case in a closed state illustrating disengagement of the latch hook in accordance with an example embodiment.

FIG. 39a is a side sectional view of a medical device case in a closed state with the clasp fully disengaged in accordance with an example embodiment.

FIG. 39b is a side sectional view of a medical device case in a closed state with the latch hook engaged in accordance with an example embodiment.

FIG. 39c is a side sectional view of a medical device case in a closed state with the latch hook and latch level engaged in accordance with an example embodiment.

FIG. 40 is a sectional view of a medical device case in a closed state with a block spacer positioned within the case in accordance with an example embodiment.

FIG. 41 is a sectional view of a medical device case in a closed state with a block spacer and medical device positioned within the case in accordance with an example embodiment.

FIG. 42 is a sectional view of a medical device case in a closed state with a block spacer and medical device positioned within the case and a tube exiting the case in accordance with an example embodiment.

FIG. 43 is a frontal sectional view of a medical device case in a closed state with a block spacer and medical device positioned within the case and a tube exiting the case in accordance with an example embodiment.

FIG. 44 is a first upper perspective view of a first exemplary spacer block in accordance with an example embodiment.

FIG. 45 is a second upper perspective view of a first exemplary spacer block in accordance with an example embodiment.

FIG. 46 is a top view of a first exemplary spacer block in accordance with an example embodiment.

FIG. 47 is a bottom view of a first exemplary spacer block in accordance with an example embodiment.

FIG. 48 is a first upper perspective view of a second exemplary spacer block in accordance with an example embodiment.

FIG. 49 is a second upper perspective view of a second exemplary spacer block in accordance with an example embodiment.

FIG. 50 is a top view of a second exemplary spacer block in accordance with an example embodiment.

FIG. 51 is a bottom view of a second exemplary spacer block in accordance with an example embodiment.

FIG. 52 is an upper perspective view of a medical device case in an opened state in accordance with an alternate embodiment in which no blocking portions are utilized.

FIG. 53 is an upper perspective view of a medical device case in an opened state with a plug installed in accordance with an alternate embodiment in which no blocking portions are utilized.

FIG. 54 is an upper perspective view of a medical device case in an opened state with a plug installed in accordance with an alternate embodiment in which no blocking portions are utilized illustrating the two positions of the plug.

FIG. 55 is an upper perspective view of a medical device case in an opened state with a plug in a resting position in accordance with an alternate embodiment in which no blocking portions are utilized.

FIG. 56 is a side view of a medical device case in a closed state in accordance with an alternate embodiment in which no blocking portions are utilized.

### DETAILED DESCRIPTION

### A. Overview.

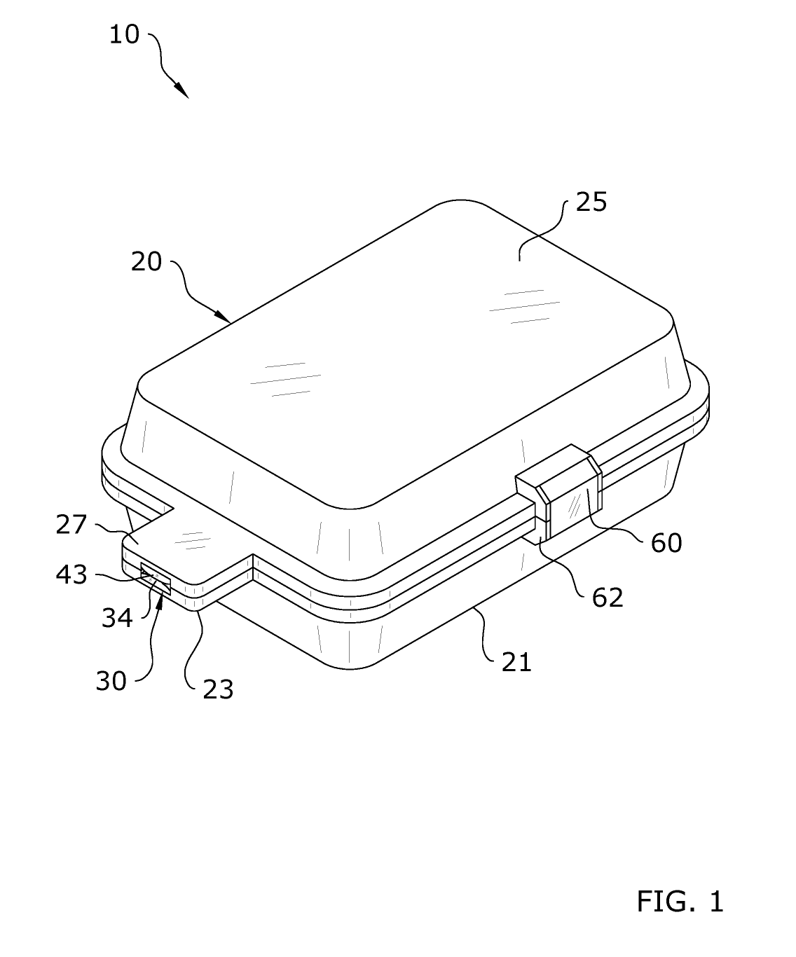

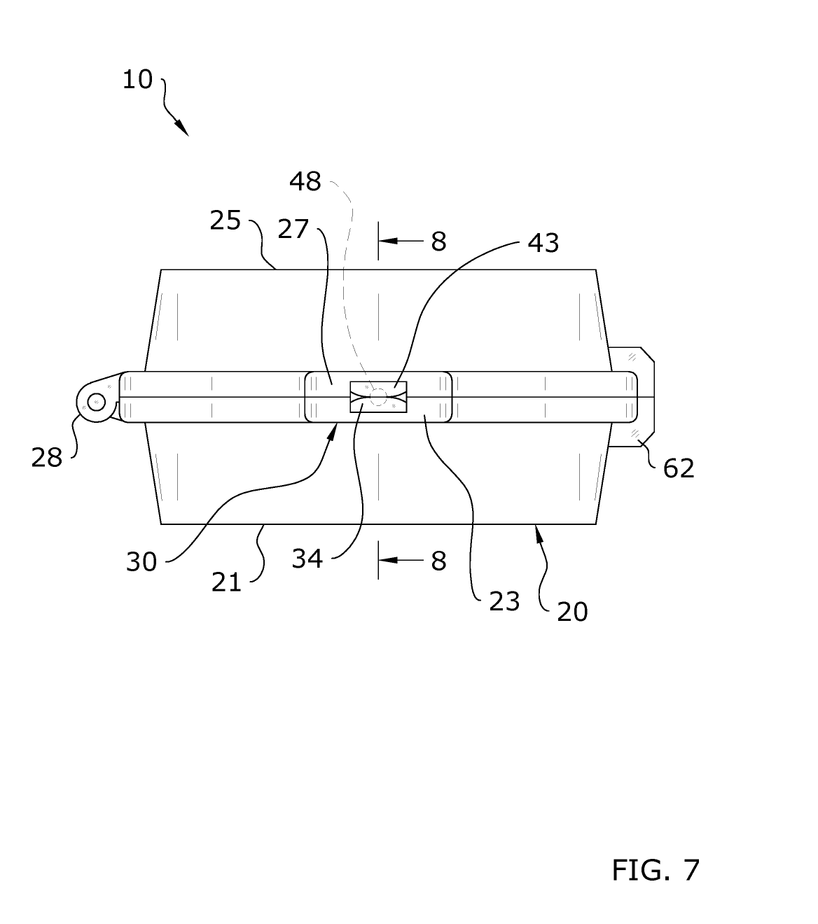

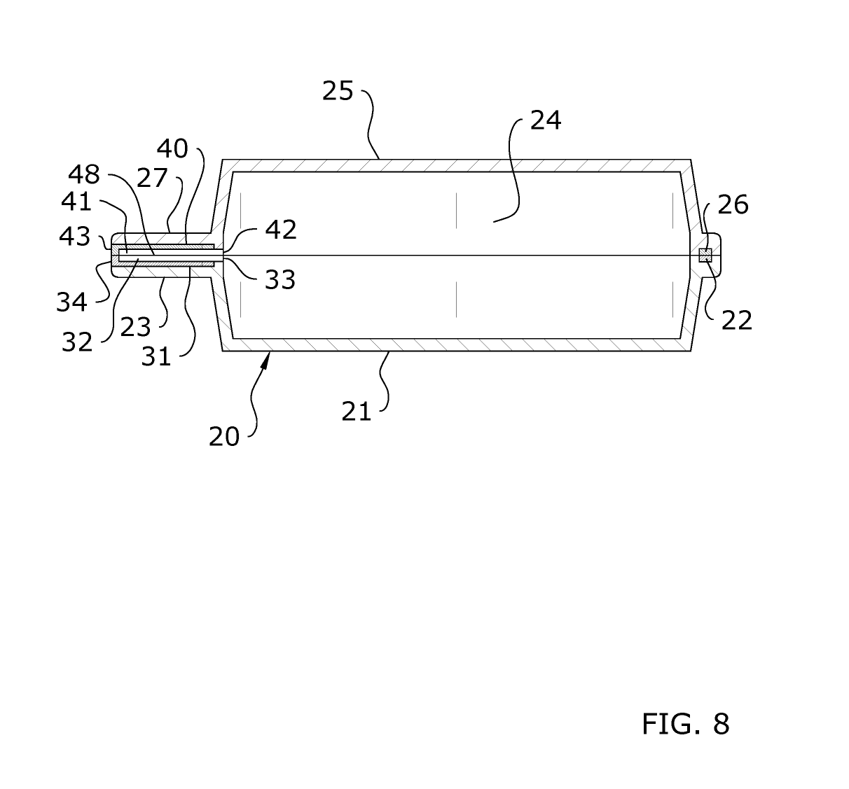

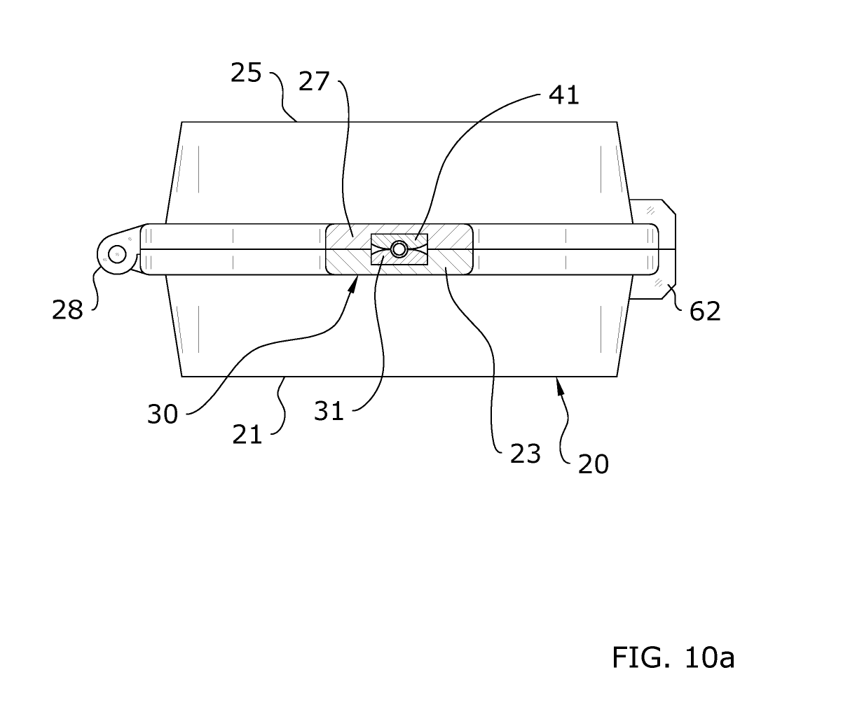

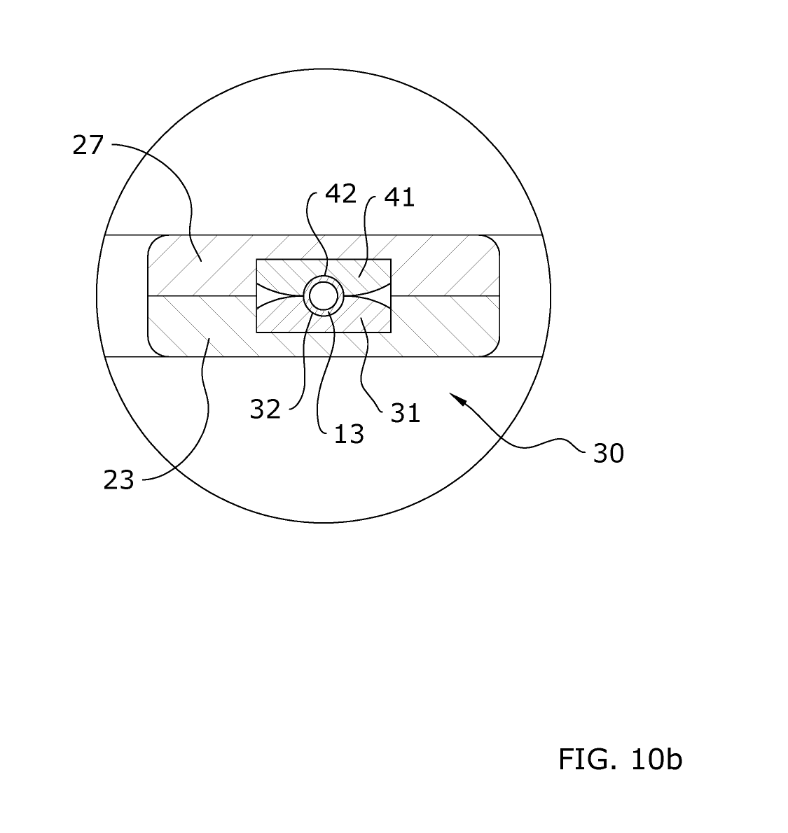

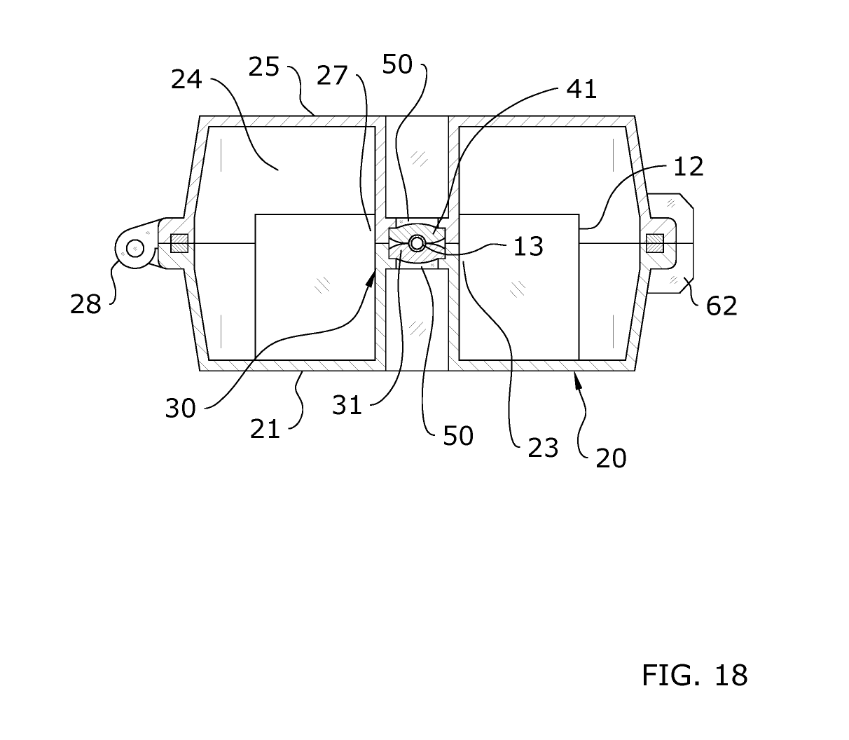

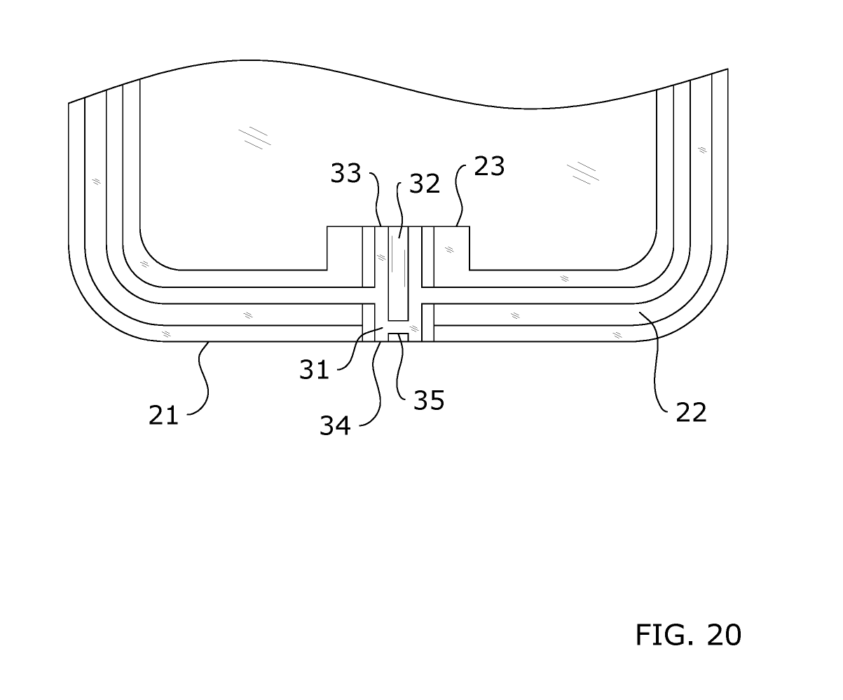

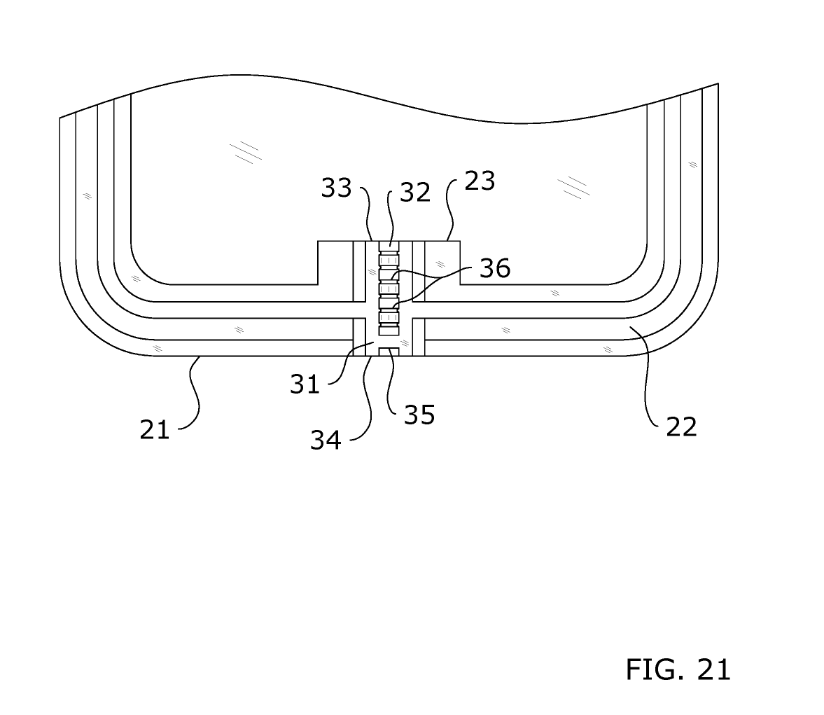

Turning now descriptively to the drawings, in which similar reference characters denote similar elements throughout the several views, FIGS. 1 through 24 illustrate a tube passage sealing system 10, which comprises a case 20 having a first portion 21 and a second portion 25 hingedly connected to the first portion 21. The first portion 21 and second portion 25 may be closed together to define a compartment 24 in which a medical device 12 may be positioned. A tube seal assembly 30 is included which will allow a tube 13 leading from the medical device 12 within the compartment 24 to exit the case 20 through a passage 48 formed in the tube seal assembly 30. The tube seal assembly 30 generally includes a first tube seal 31 and second tube seal 40 which combine when the case 20 is closed to seal around the tube 13. Some embodiments may include blocking portions 35, 44 on the tube seals 31, 40 which will act to seal the passage 48 when a tube 13 is not present.

It should be noted that the present invention may be utilized with a wide range of devices, such as medical equipment, networking equipment, electrical devices, pumps, meters, sampling valves, and the like. While a focus is on medical devices 12, any type of device which includes a conduit or tube 13 may be utilized in combination with the present invention to protect the device from fluid intrusion.

For example, medical equipment could include infusion pumps, peristaltic pumps, monitors, and electrical stimulus devices. Networking equipment could include routers, switches and the like. Electrical devices could include electrical relays, programmable logic controllers, timers, and the like. The figures and description herein should thus not be construed as limiting usage of the present invention to medical devices 12.

### B. Case.

As shown throughout the figures, the present invention generally comprises a case 20 having a first portion 21 and a second portion 25 which close to form a compartment 24 adapted to sealably store and retain a medical device 12 therein. The case 20 generally has an open state in which its compartment 24 is accessible to place a medical device 12 therein. The case 20 also has a closed state in which the first and second portions 21, 25 are closed against each other. When in a closed state, the compartment 24 is sealed so as to prevent escape of any fluids from the medical device 12 and to prevent ingress of fluids into compartment 24.

The first portion 21 and second portion 25 are preferably hingedly secured to each other via a hinge 28 as shown in the figures, though it is appreciated that other configurations may be utilized to position the case 20 between its open state and its closed state. The present invention should not be construed as limited to hinged configurations. For example, one embodiment may include completely separable first and second portions 21, 25.

The case 20 may be comprised of any variety of shapes, sizes and configurations to accommodate a wide range of medical devices 12. While the figures illustrate the case 20 as being generally rectangular in shape, it should be appreciated that the case 20 could be any number of shapes, including round, triangular, polygonal and the like. Larger cases 20 may be provided for larger medical devices 12 and smaller cases 20 may be provided for more compact medical devices 12.

The case 20 may include a handle as shown or in some embodiments may omit a handle entirely. In some embodiments, the case 20 may include a lock to prevent unauthorized access to the medical device 12 stored therein. The case 20 may also include a clasp, buckle, connector, fastener, or other devices to removably or lockably secure the first and second portions 21, 25 together to seal the compartment 24.

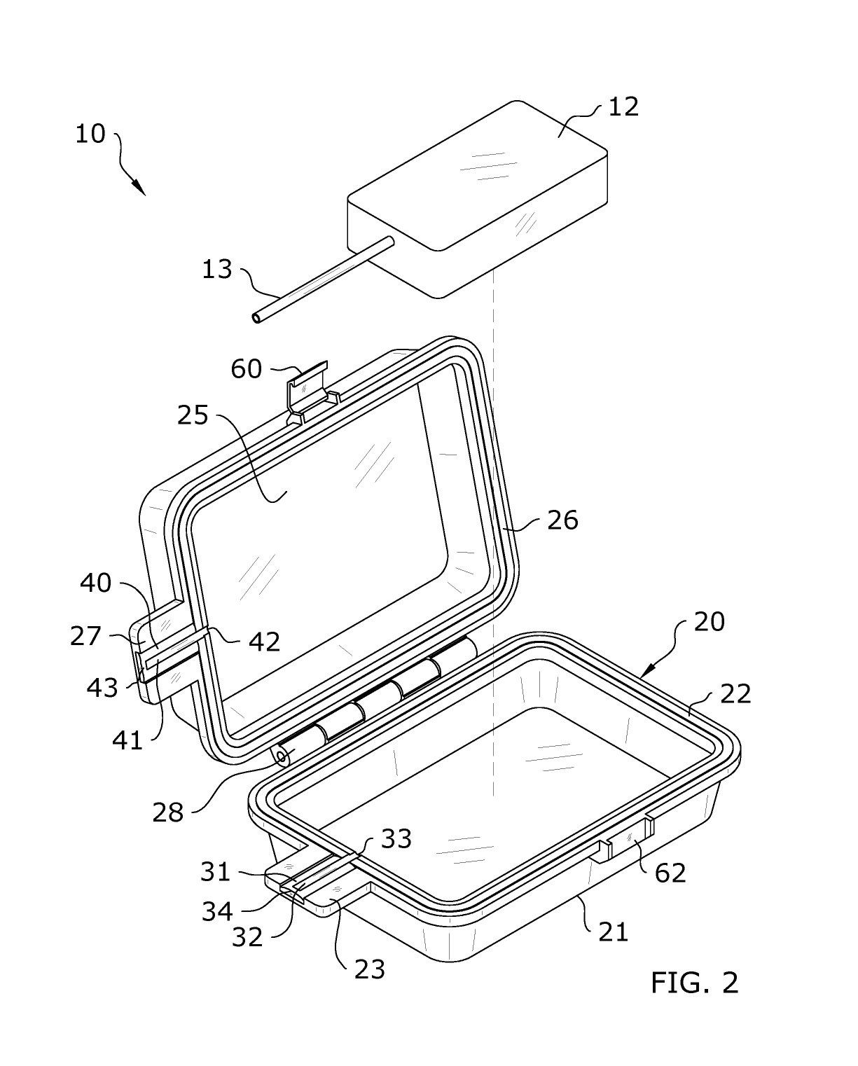

The figures illustrate a clasp 60 and clasp anchor 62 being used to removably secure the first portion 21 to the second portion 25 to close the case 20. As best shown in FIG. 2, a clasp 60 is positioned on the second portion 25 of the case 20 and a corresponding clasp anchor 62 is positioned on the first portion 21 of the case 20. When the case 20 is closed, the clasp 60 may removably engage with the clasp anchor 62 to secure the case 20 in its closed position.

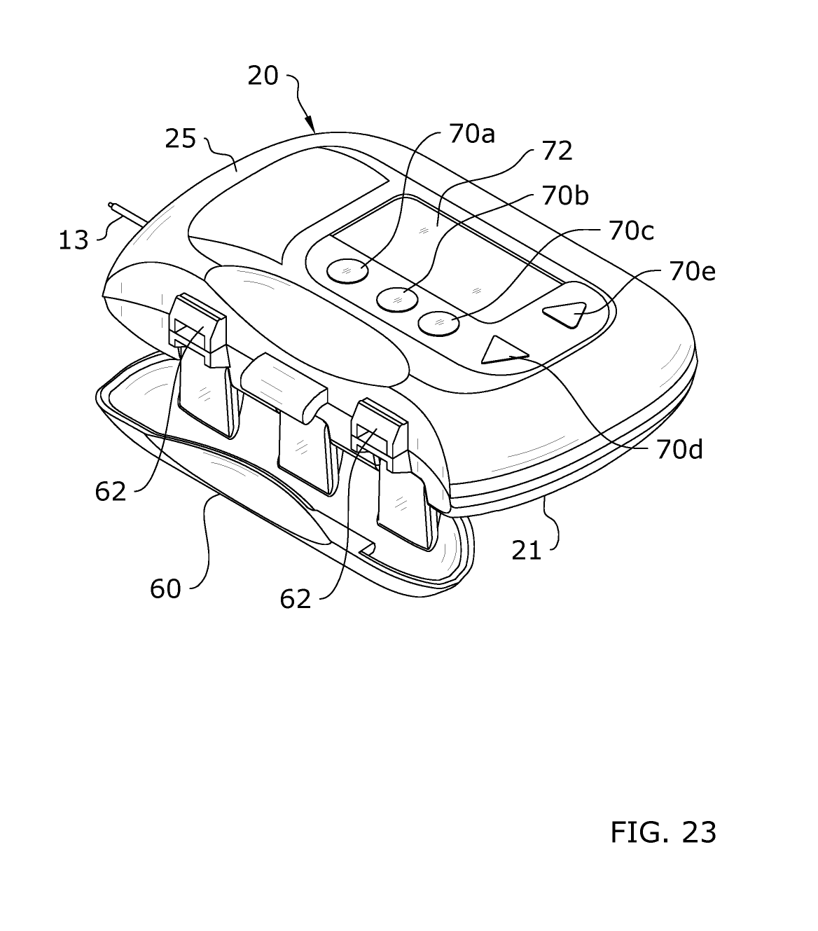

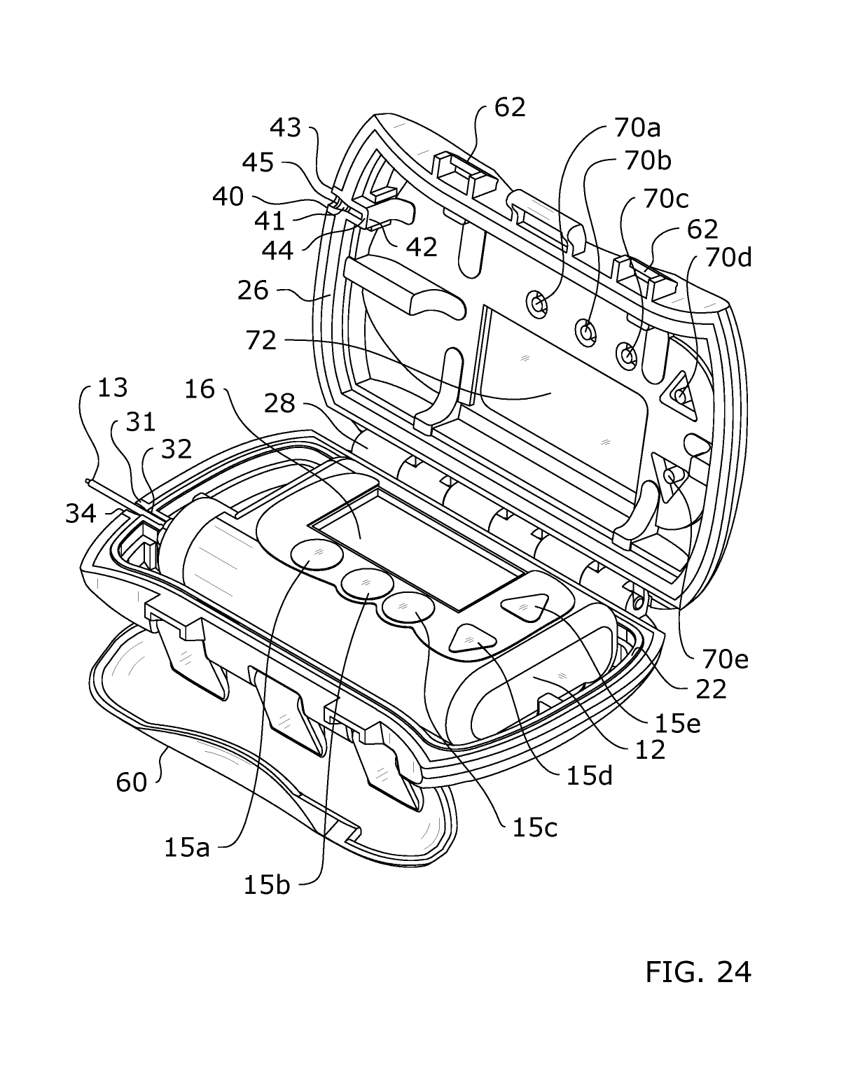

It should be appreciated that any method or device known in the art for removably securing a first portion 21 to a second portion 25 of a case 20 may be utilized. For example, FIGS. 23 and 24 illustrate a more complex clasp 60 on the first portion 21 of the case 20 and a clasp anchor 62 on the second portion 25 of the case 20.

The first portion 21 and second portion 25 are preferably similar in shape and size so as to form a tight seal against each other when closed. In a preferred embodiment as shown in FIG. 2, the first portion 21 will include a first perimeter seal 22 along its inner perimeter and the second portion 25 will include a second perimeter seal 26 along its inner perimeter.

The perimeter seals 22, 26 may vary in construction, but will generally be comprised of a sealing material such as rubber or the like which extends around the inner perimeter of the first portion 21 and second portion 25 of the case 20. When the case 20 is in its closed state, the first perimeter seal 22 will press and seal against the second perimeter seal 26. The perimeter seals 22, 26 may be transparent or partially transparent in some embodiments.

The perimeter seals 22, 26 will act to press against each other in a manner which fully seals the compartment 24 of the case 20 when the case 20 is closed by engaging the first portion 21 with the second portion 25. Thus, fluids will be prevented from entering the compartment 24 via ingress between the first and second portions 21, 25.

Main embodiments shown in the figures utilize a two-element seal system for both the tube seal assembly 30 and the perimeter seals 22, 26 (i.e., the first and second tube seals 31, 40 and the perimeter seals 22, 26 each have two elements). While this is a preferred embodiment, it may add unwanted expense or complexity to the present invention. A partial two-element seal may thus have cost and simplicity advantages to the full two-element seals shown in main embodiments.

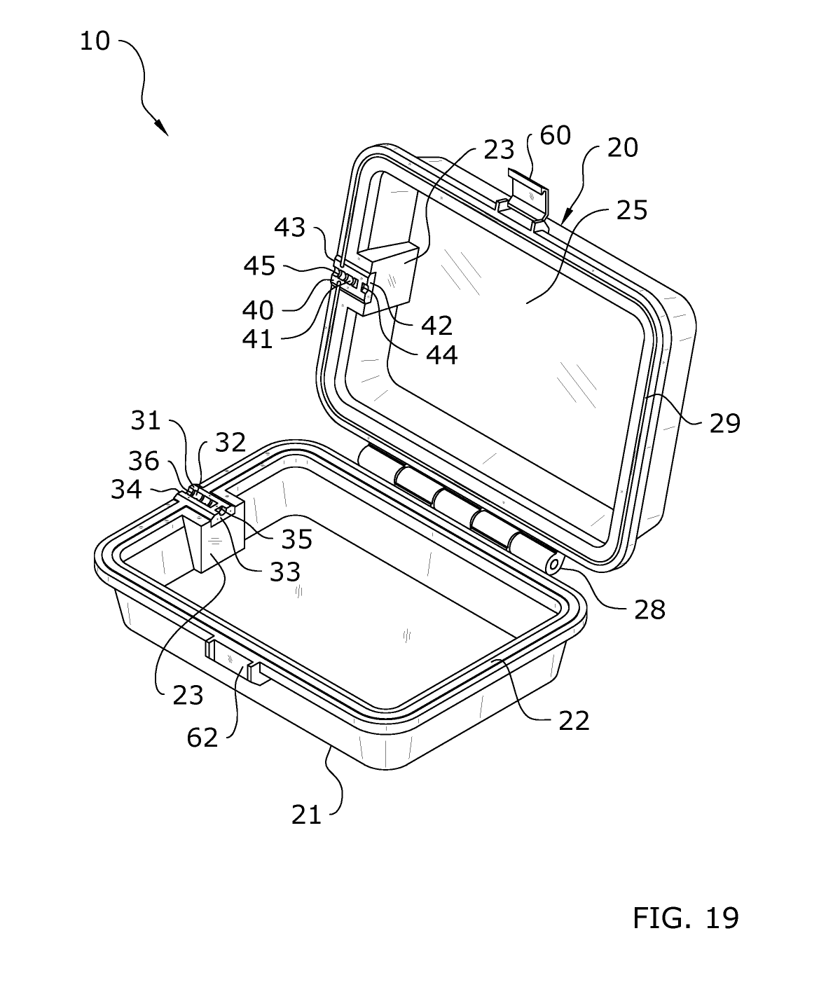

FIG. 19 illustrates an alternate embodiment of the present invention in which only one perimeter seal 22 is utilized. A majority of the perimeter of the case 20 is sealed by a traditional single element seal and only in the area of the passage 48 for the tube 13 is a two-element seal utilized. In this embodiment, the first portion 21 of the case 20 includes a first perimeter seal 22 as with the main embodiment. However, the second portion 25 of the case 20 includes a perimeter engagement member 29 instead of a second perimeter seal 26.

The perimeter engagement member 29 is comprised of a raised protrusion which extends around the outer perimeter of the second portion 25 of the case 20 and which engages with and indents the first perimeter seal 22 of the first portion 21 to effectuate a seal of the majority of the perimeter of the case 20. As the seal 22 gets near the tube assembly 30, the second tube seal 40 of the second portion 25 of the case 20 overlaps the perimeter engagement member 29 to ensure sealing at the transition between the perimeter seal 22 and the tube seal assembly 30. It should be appreciated that, in other embodiments, the perimeter engagement member 29 may be included on the first portion 21 of the case 20 and used in combination with a second perimeter seal 26 on the second portion 25 instead.

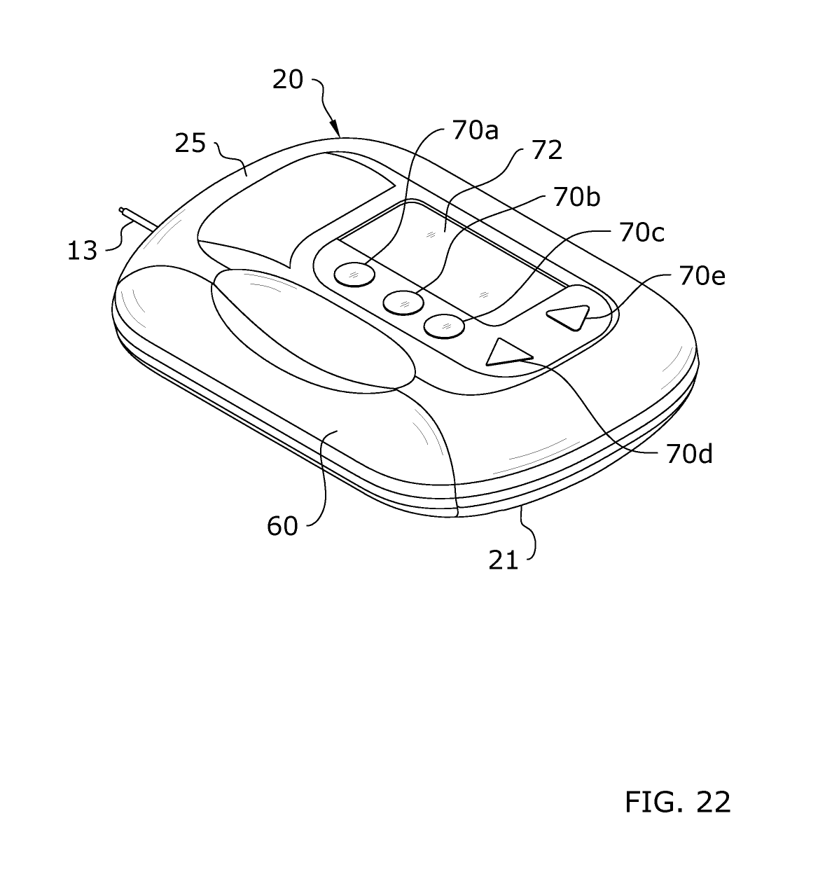

As noted, the structure of the case 20 may vary in different embodiments. FIGS. 22-24 illustrate an alternate embodiment of the case 20 which is adapted to include pass-through controls 70a-e to allow control of the device controls 15a-e of the medical device 12. For example, a first pass-through control 70a of the case 20 will be positioned directly above a corresponding device control 15a such that pressing down on the control 70a on the case 20 will cause the underlying device control 15a to be depressed.

The number, orientation, and positioning of the controls 70a-e may vary in different embodiments and should not be construed as limited by the exemplary figures. The alternate embodiment of the case 20 may also include a window 72 which allows a user to view the device display 16 of the medical device 12 when the case 20 is closed.

### C. Tube Seal Assembly.

The present invention is adapted to allow the tube 13 of a medical device 12 stored within the compartment 24 of the case 20 to exit the case 20 in a manner which seals the compartment 24 whether the tube 13 is present or absent. To provide such a feature, the present invention includes a tube seal assembly 30 which is shown throughout the figures and generally comprises a first tube seal 31 on the first portion 21 of the case 20 and a second tube seal 40 on the second portion 25 of the case 20.

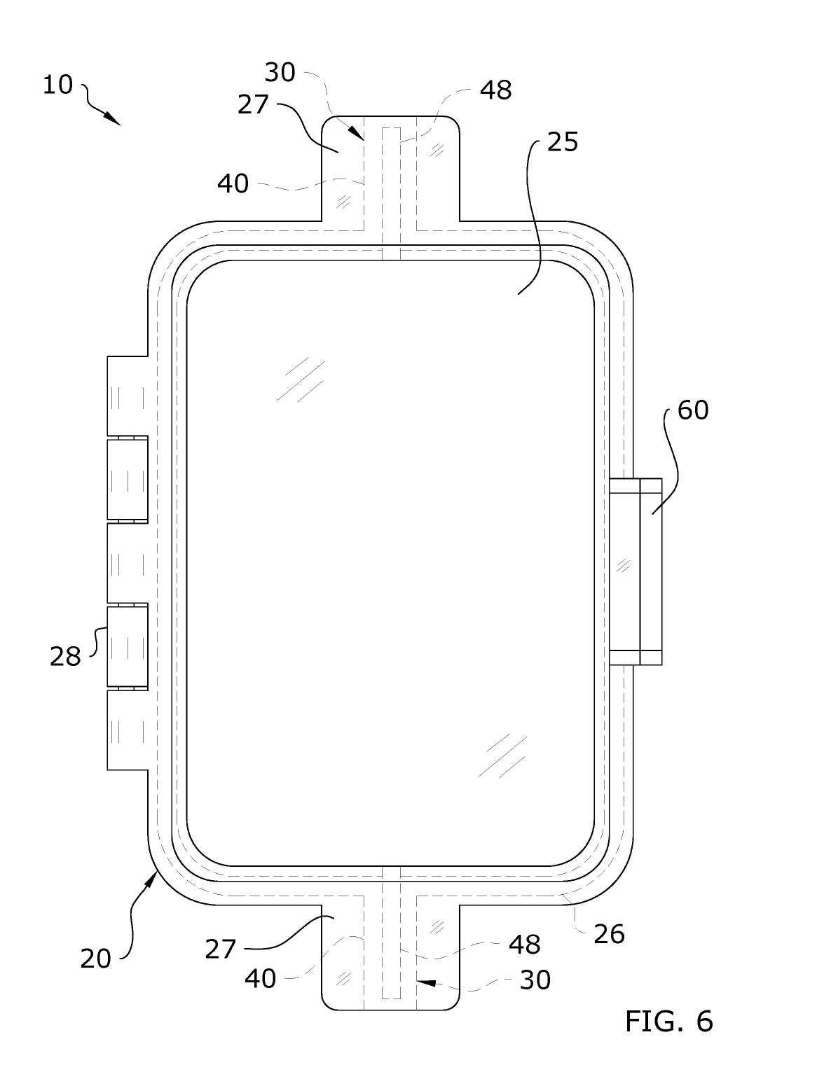

In some embodiments such as shown in FIG. 6, the case 20 may include multiple tube seal assemblies 30. For example, such an embodiment may be utilized with certain electrical devices such as probes which may include two or more tubes 13, such as a power cord and a measuring probe. Other embodiments, such as a case 20 adapted for use with a router, may include tube seal assemblies 30 to accommodate multiple tubes 13 entering or exiting the case 20.

As best shown in FIGS. 2, 3, and 18, the tube seal assembly 30 may comprise a first tube seal 31 positioned on the first portion 21 of the case 20 and a second tube seal 40 positioned on the second portion 25 of the case 20. Each tube seal 31, 40 generally comprises a sealing material such as rubber, with the first tube seal 31 adapted to press and seal against the second tube seal 40 when the case 20 is closed. The tube seals 31, 40 may be transparent or partially transparent in some embodiments. In some embodiments, the first tube seal 31 may be integrally formed with the first perimeter seal 22 and the second tube seal 40 may be integrally formed with the second perimeter seal 26.

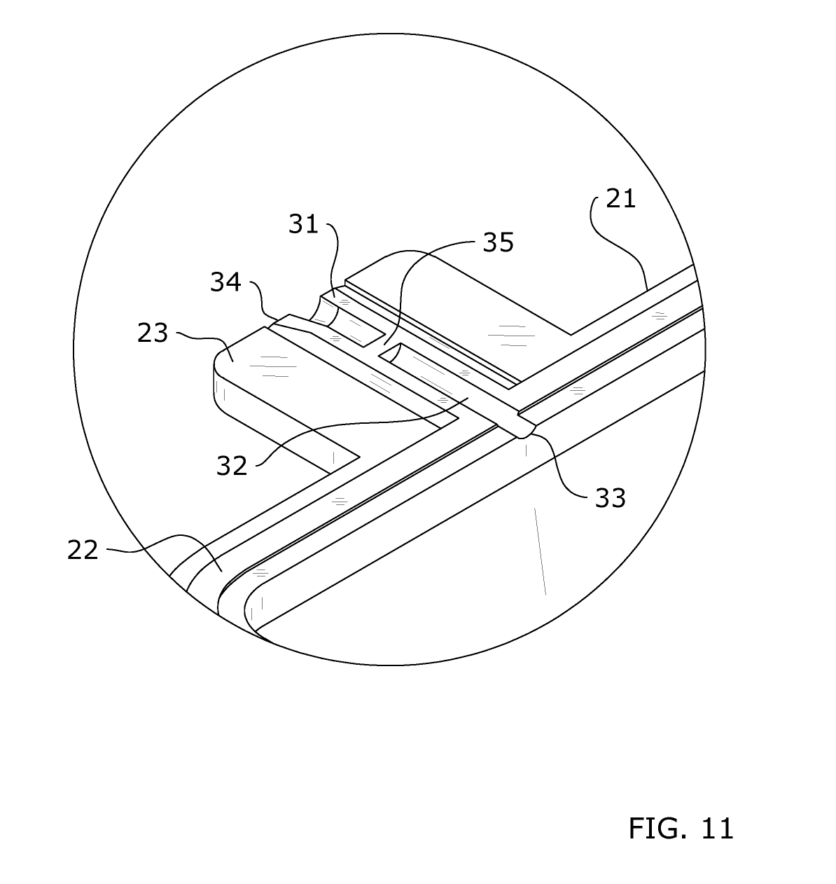

The positioning of the tube seals 31, 40 within the case 20 may vary in different embodiments. Preferred embodiments will utilize a pair of mounts 23, 27 comprised of extended sections of the case 20 which include the tube seals 31, 40. As shown in FIG. 2, the first portion 21 of the case 20 will generally include a first mount 23 and the second portion 25 of the case 20 will generally include a second mount 27. FIGS. 1-9 and 11 illustrate an embodiment in which the mounts 23, 27 extend outwardly from the perimeter of the case 20. FIGS. 12-21 illustrate an embodiment in which the mounts 23, 27 extend inwardly from the perimeter of the case 20.

The tube seals 31, 40 are each generally comprised of a rectangular or similarly-shaped member with a channel 32, 41 formed in the member which extends perpendicularly with respect to the perimeter of the case 20. The material used in the tube seals 31, 40 may vary in different embodiments, but will preferably be resilient so as to bulge outward when the blocking portions 35, 44 are deformed around a tube 13.

The first tube seal 31 will generally include a first channel 32 and the second tube seal 40 will generally include a second channel 41 as best shown in FIG. 2. When the case 20 is closed, the first channel 32 and second channel 41 will combine to form a passage 48 through which the tube 13 of the medical device 12 may extend to exit the compartment 24 of the case 20.

To prevent ingress of fluids into the case 20 when no tube 13 is present, the first tube seal 31 will generally include a first blocking portion 35 and the second tube seal 40 will generally include a second blocking portion 44. The blocking portions 35, 44 will press and seal against each other when the case 20 is in its closed state to seal the passage 48 and prevent fluid escapement. Thus, the first blocking portion 35 will preferably be aligned with the second blocking portion 44 such that they contact each other when the case 20 is closed.

Each blocking portion 35, 44 is generally comprised of a flexible, resilient material such as rubber which extends across each respective channel 32, 41 of the tube seals 31, 40. The size, shape, and configuration of the blocking portions 35, 44 may vary in different embodiments so long as they act to seal the passage 48 when no tube 13 is present and the case 20 is closed.

When a tube 13 is installed in the case 20, the passage 48 is sealed by the exterior diameter of the tube 13 compressing material in the unblocked portion of the passage 48. Because the passage 48 is sealed against the tube 13 body, the blocking portions 35, 44 no longer must seal and can therefore tolerate significant deformation from deforming around the tube 13 body.

The blocking portions 35, 44 may be positioned differently in different embodiments of the present invention. FIGS. 2, 11, 12, and 13 illustrate alternate positioning of the blocking portions 35, 44. FIG. 2 illustrates an embodiment in which the first blocking portion 35 is positioned at the distal end 34 of the first channel 32 and the second blocking portion 44 is positioned at the distal end 43 of the second channel 41.

FIG. 11 illustrates an alternate embodiment in which the blocking portions 35, 44 are positioned at approximately 40% of the length between the distal ends 34, 43 and proximal ends 33, 42 of the respective channels 32, 41. FIG. 12 illustrates an embodiment in which the blocking portions 35, 44 are positioned adjacent to the proximal ends 33, 42 of the respective channels 32, 41. FIG. 13 illustrates an embodiment in which the blocking portions 35, 44 are positioned adjacent to the distal ends 34, 43 of the respective channels 32, 41.

Any configuration may be utilized, and it should be appreciated that the positioning of the blocking portions 35, 44 may vary widely between different embodiments. The scope of the present invention should not be construed as being limited by the exemplary figures. The blocking portions 35, 44 could be positioned at any position between and including the respective ends 33, 34, 42, 43 of the respective channels 32, 41.

The interior walls of the tubular channels 32, 41 of the tube seals 31, 40 may be round and smooth as best shown in FIGS. 11-13. While this configuration may work for most uses, it may also require precise tolerances on the tube seal 31, 40 outer diameters and on the tubular channel 32, 41 area of each seal 31, 40. Non-foam elastomers are similar to hydraulic fluid in that they are incompressible and when a seal is compressed the compressed material must bulge out somewhere else.

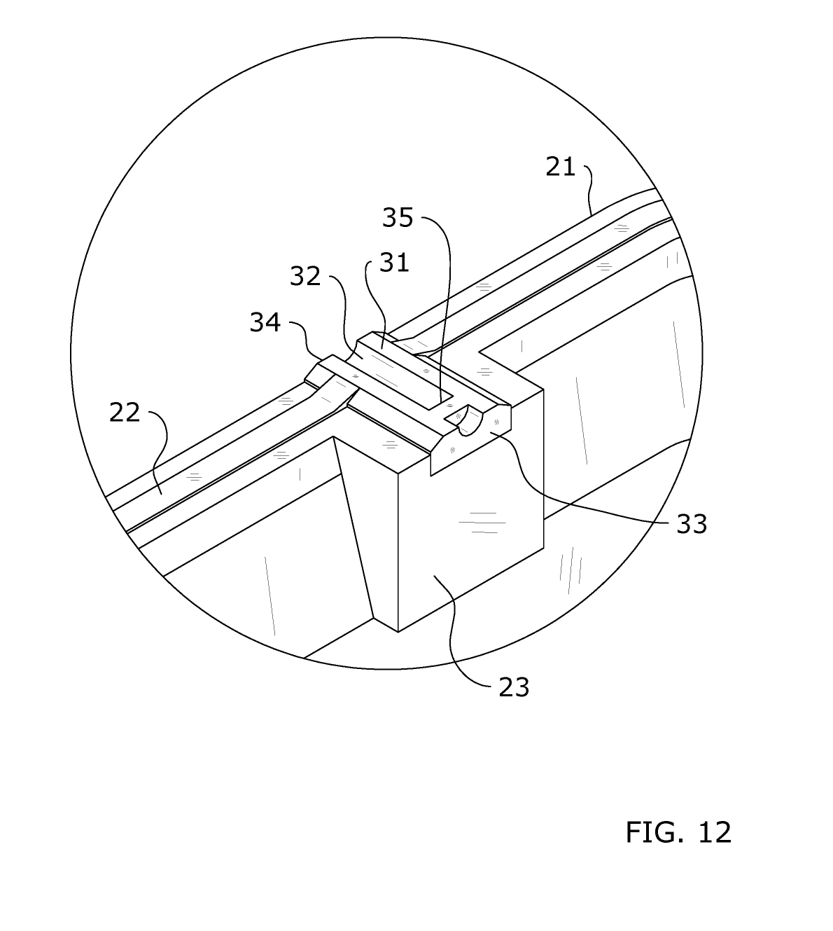

To alleviate this potential problem, some embodiments of the present invention will include a ribbed profile as best shown in FIGS. 14, 15, and 18. In such embodiments, first ribs 36 will be positioned within the first channel 32 and second ribs 45 will be positioned within the second channel 41. Each rib 36, 45 is generally comprised of a raised elongated portion extending within the inner circumference of the channels 32, 41.

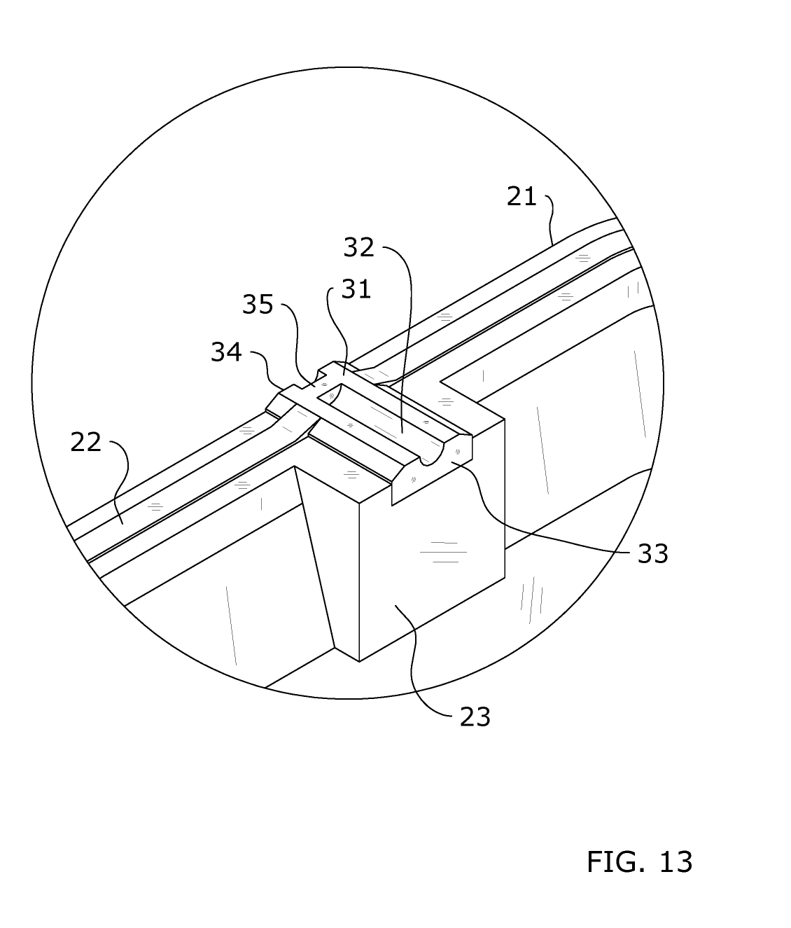

FIG. 14 illustrates a first channel 32 having closely-spaced first ribs 36. When the tips of the ribs 36 are compressed by the tube 13, the seal material can bulge out into the spaces between the ribs 36. This will allow a lower closing force than if the entire main seal 31, 40 body had to be compressed to seal against the tube 13. FIG. 15 illustrates a first channel 32 having more space between the first ribs 36. This configuration will function similarly to the previously-described embodiment but with the added benefit of the redundancy provided by multiple independent sealing areas.

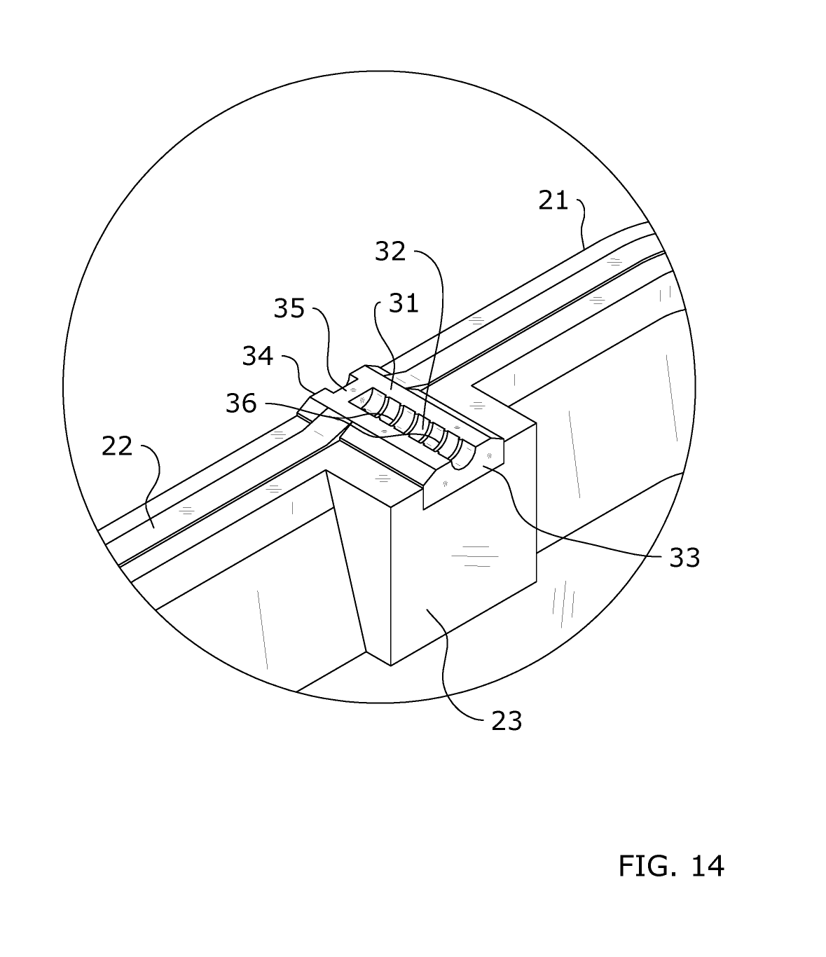

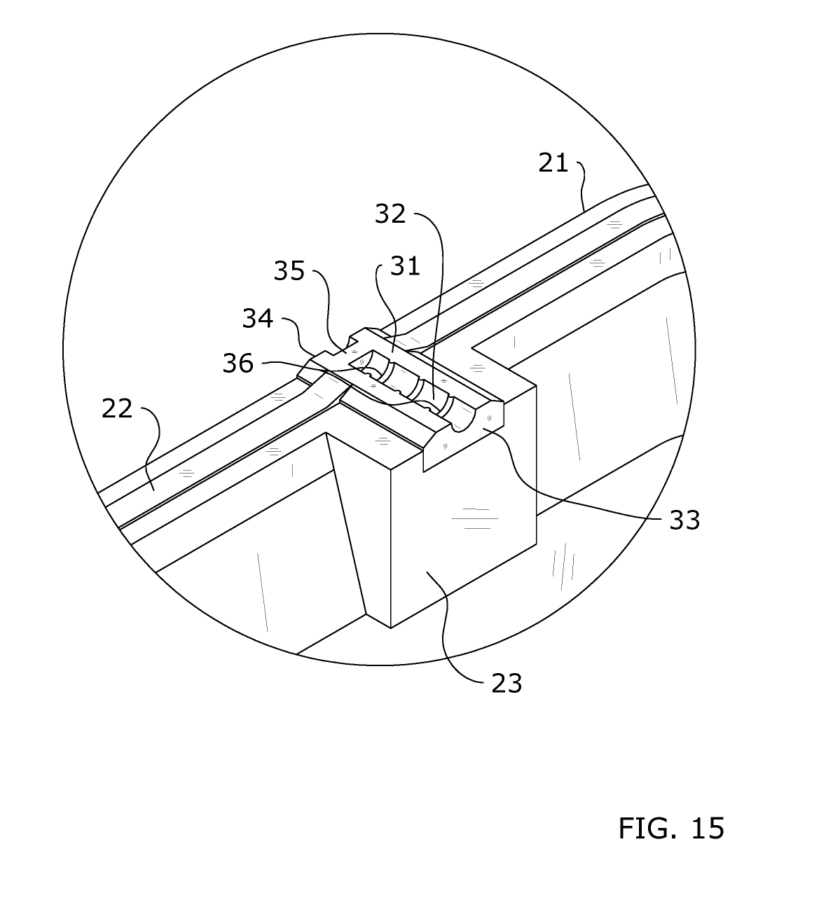

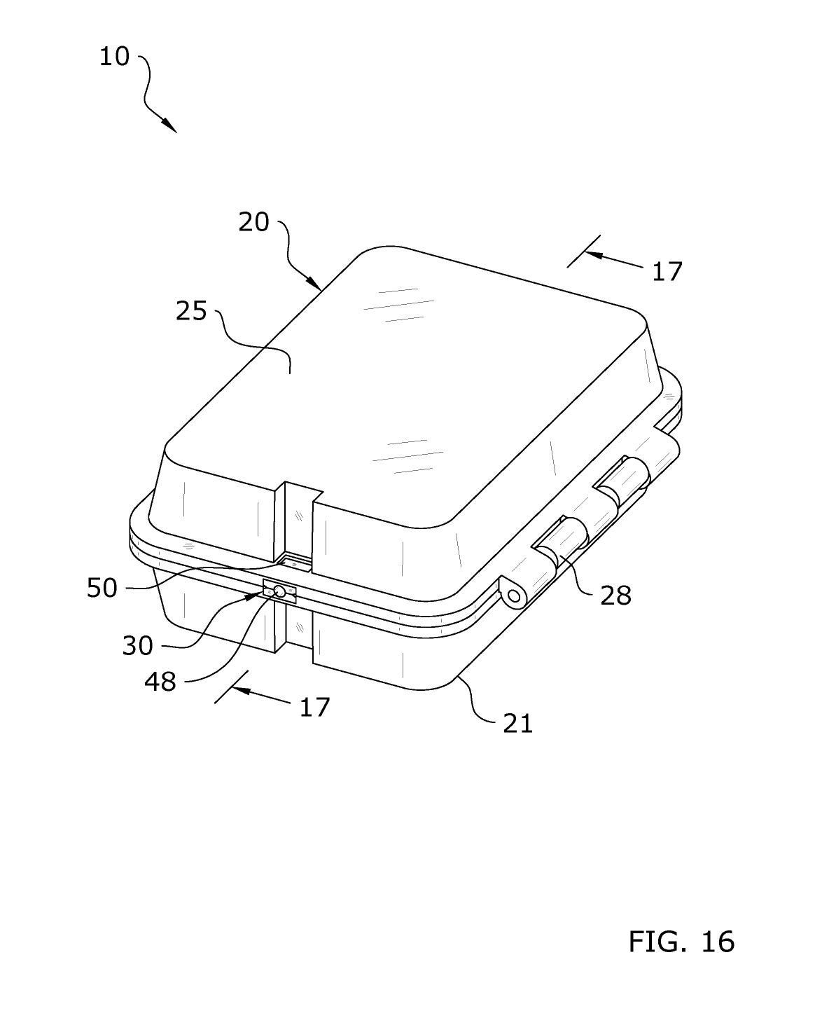

FIG. 16 illustrates an embodiment in which the case includes a displacement opening 50 aligned with the tube seal assembly 30. This feature may be included in embodiments where the tube 13 wall is more flexible but not stiff enough to displace the blocking portions 35, 44 of the seals 31, 40 without collapsing the tube 13 wall itself. The displacement opening 50 allows the blocking portions 35, 44 to be displaced therein instead of simply compressing the material of the tube 13 to prevent free flow of fluid there through.

### D. Operation of Preferred Embodiment.

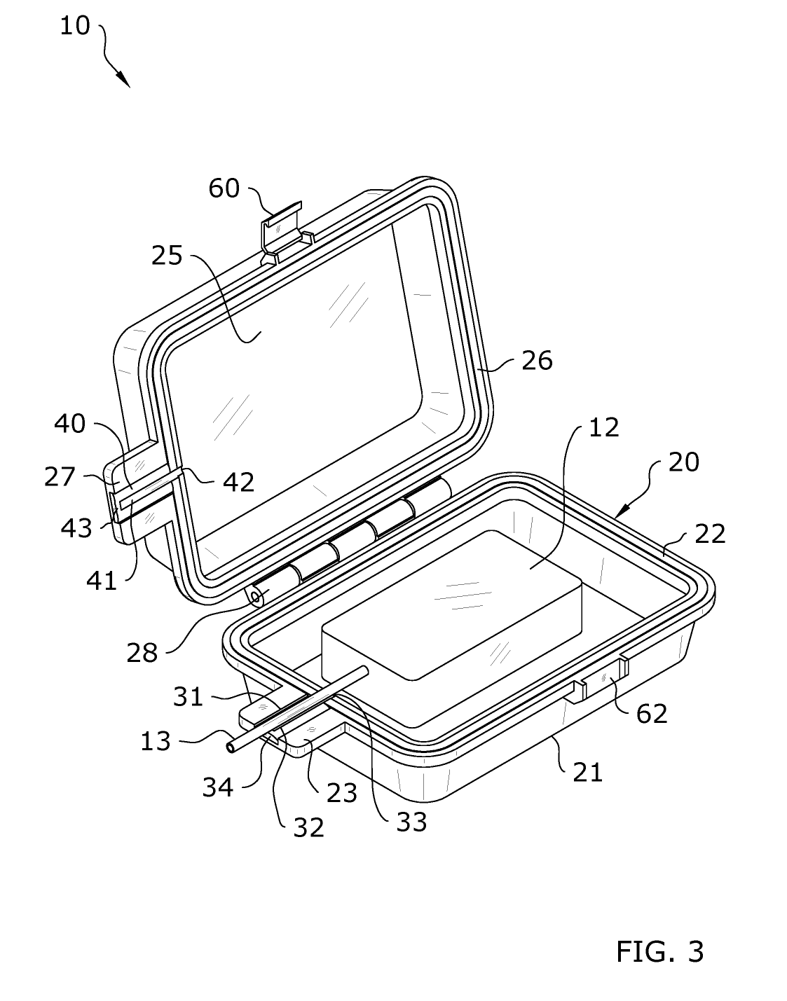

In use, the case 20 is first placed into its opened state by separating its first portion 21 from its second portion 25 to expose its compartment 24. The medical device 12 may then be positioned within the compartment 24 of the case 20 as shown in FIGS. 2 and 3. The medical device 12 may be secured in place within the compartment 24 through any methods or devices known to secure a device within a case 20.

The present invention may be utilized with any type of medical device 12 that includes a tube 13 or other conduit extending therefrom and which would benefit from being protected by a case 20. For example and without limitation, the medical device 12 could be comprised of an insulin pump, which generally utilizes a tubing system which extends from an insulin reservoir and interfaces with a patient's body.

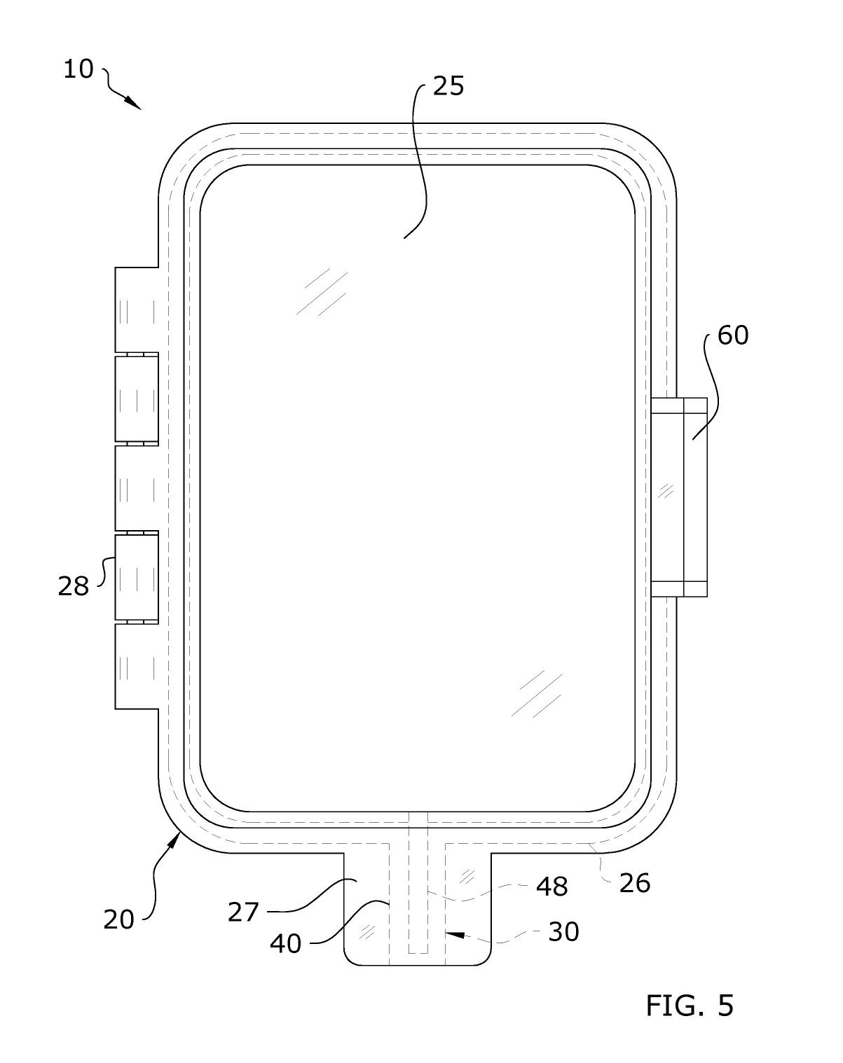

With the medical device 12 positioned within the compartment 24, the case 20 may be placed into its closed state to protect the medical device 12 as shown in FIG. 5. The tube 13 of the medical device 12, if present, will be positioned within the first channel 32 of the first tube seal 31 so as to extend out of the case 20 prior to its closing.

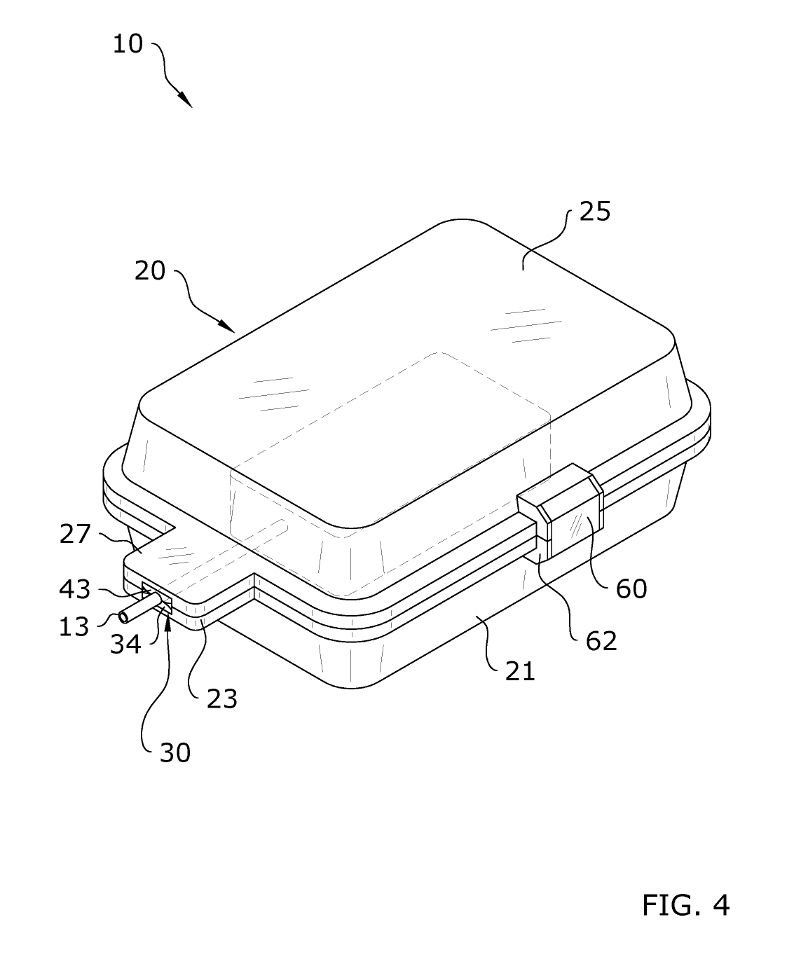

The first and second portions 21, 25 of the case 20 may then be closed together to encapsulate the medical device 12 within the compartment 24. When the case is in its closed state, the perimeter seals 22, 26 and/or perimeter engagement member 29 will act to seal the compartment 24 and prevent intrusion of fluids into the compartment 24 of the case 20 whether a tube 13 extends out of the compartment 24 or not. In some embodiments, the portions 21, 25 may be secured or locked to each other to prevent inadvertent opening of the case 20. When closed, the first channel 32 of the first tube seal 31 and the second channel 41 of the second tube seal 40 will combine to form a passage 48 through which the tube 13, if present, extends out of the case 20. FIG. 4 best illustrates a tube 13 of a medical device 12 extending out of the compartment 24 of the case 20 through the passage 48.

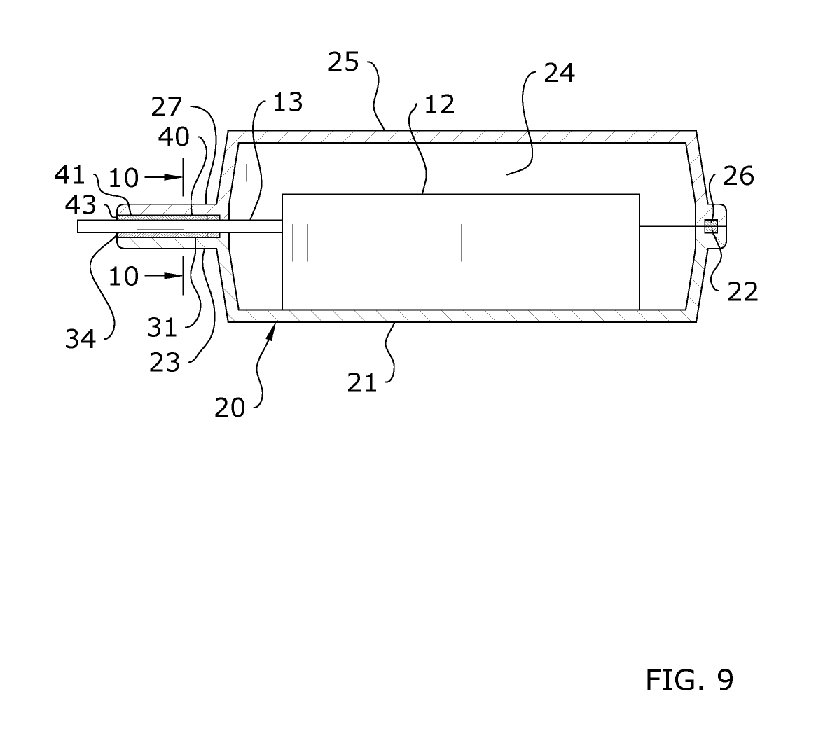

FIG. 9 shows a tube 13 extending through the passage 48 in an embodiment which omits blocking portions 35, 44. The tube seal assembly 30 will prevent leakage through the first and second tube seals 31, 40 pressing and sealing around the body of the tube 13 when the tube 13 is present. However, the passage 48 will remained open absent the tube 13, which may allow fluid intrusion when a tube 13 is not present in the passage 48.

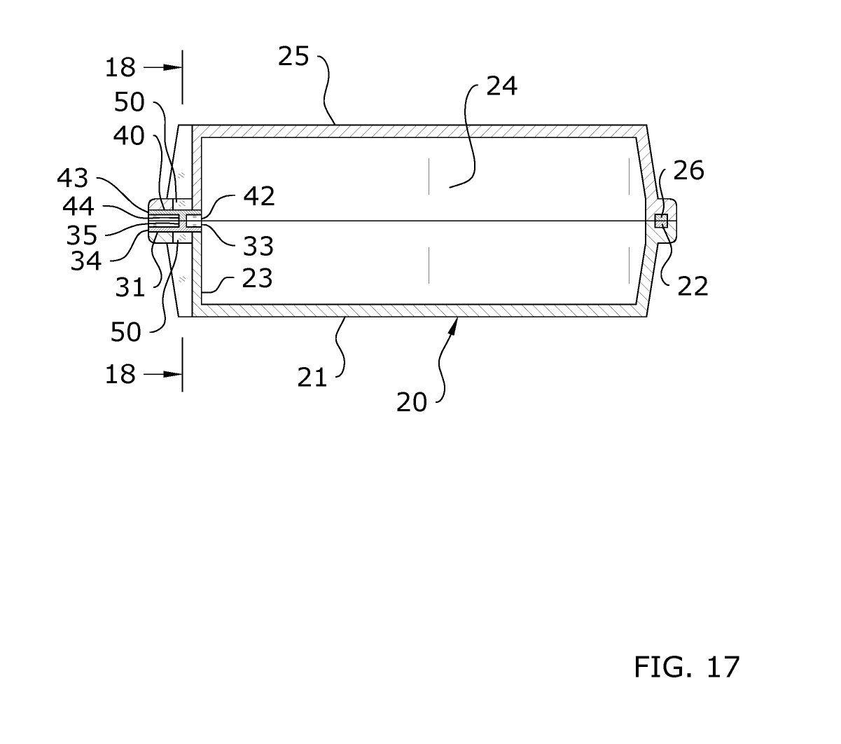

To alleviate this potential problem, some embodiments of the present invention may utilize one or more blocking portions 35, 44. The blocking portions 35, 44 will act to close the passage 48 when a tube 13 is not present therein. FIG. 17 best illustrates the sealing of the passage 48 absent the presence of a tube 13 therein in such embodiments which utilize blocking portions 35, 44. When the case 20 is closed without a tube 13, the blocking portions 35, 44 will press against each other to close the passageway 48. This will seal the compartment 24 and prevent fluid leakage into or out of the compartment 24.

### E. Medical Device Case.

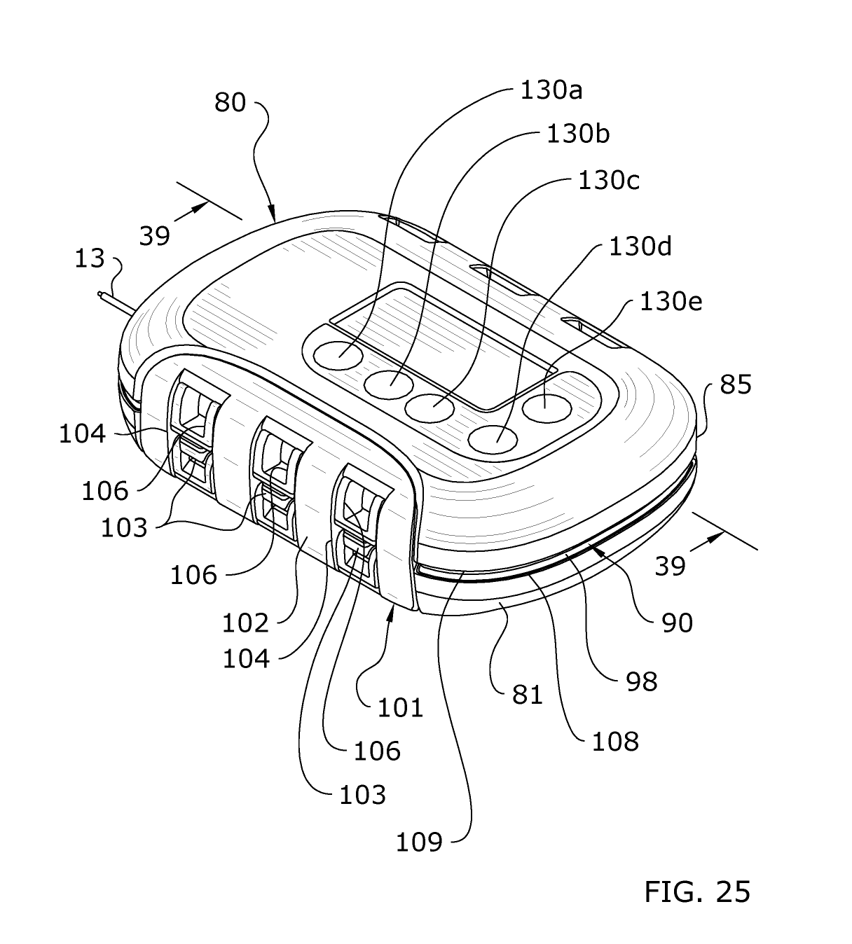

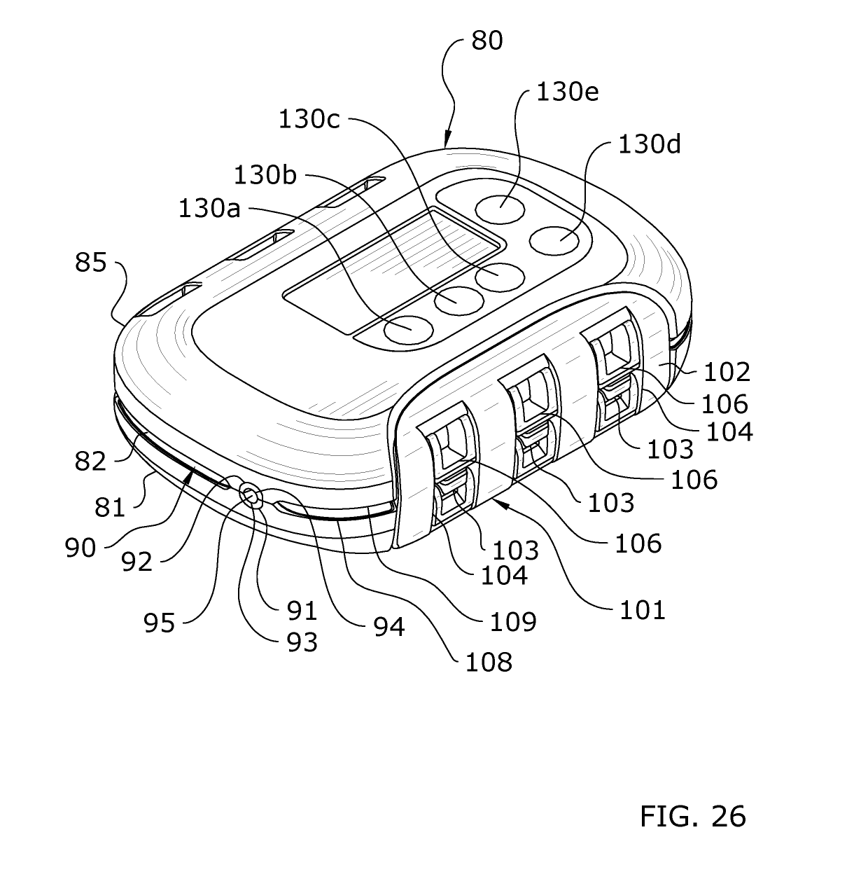

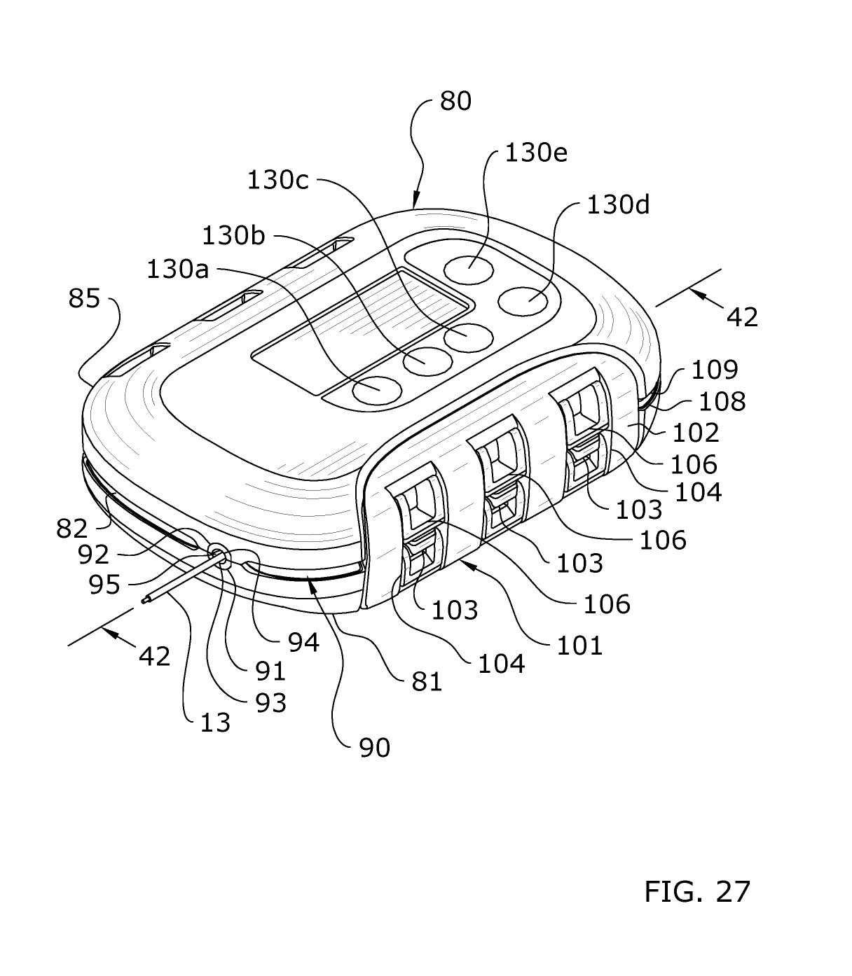

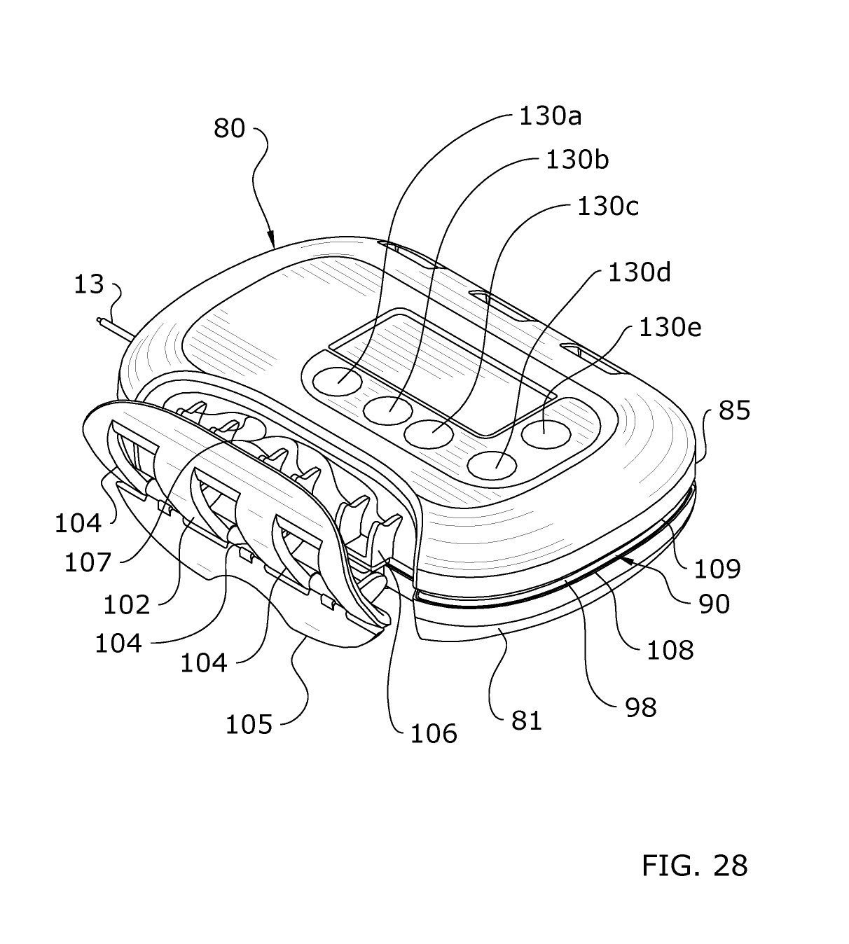

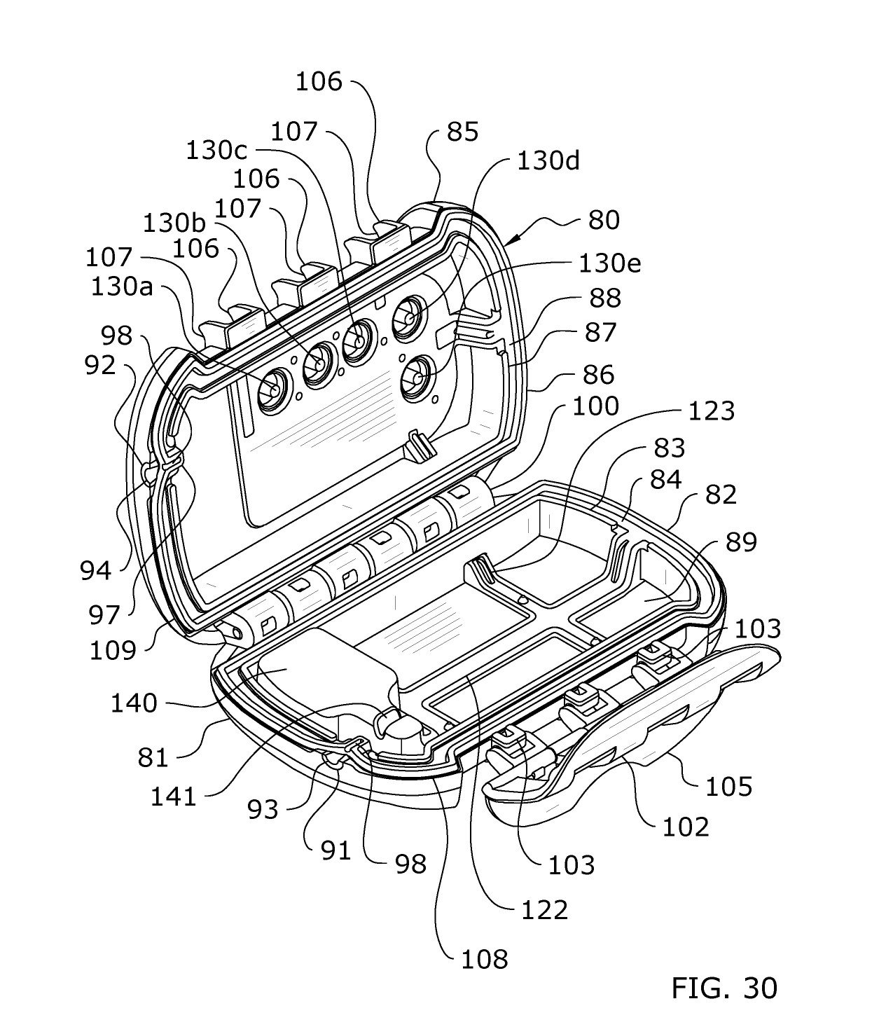

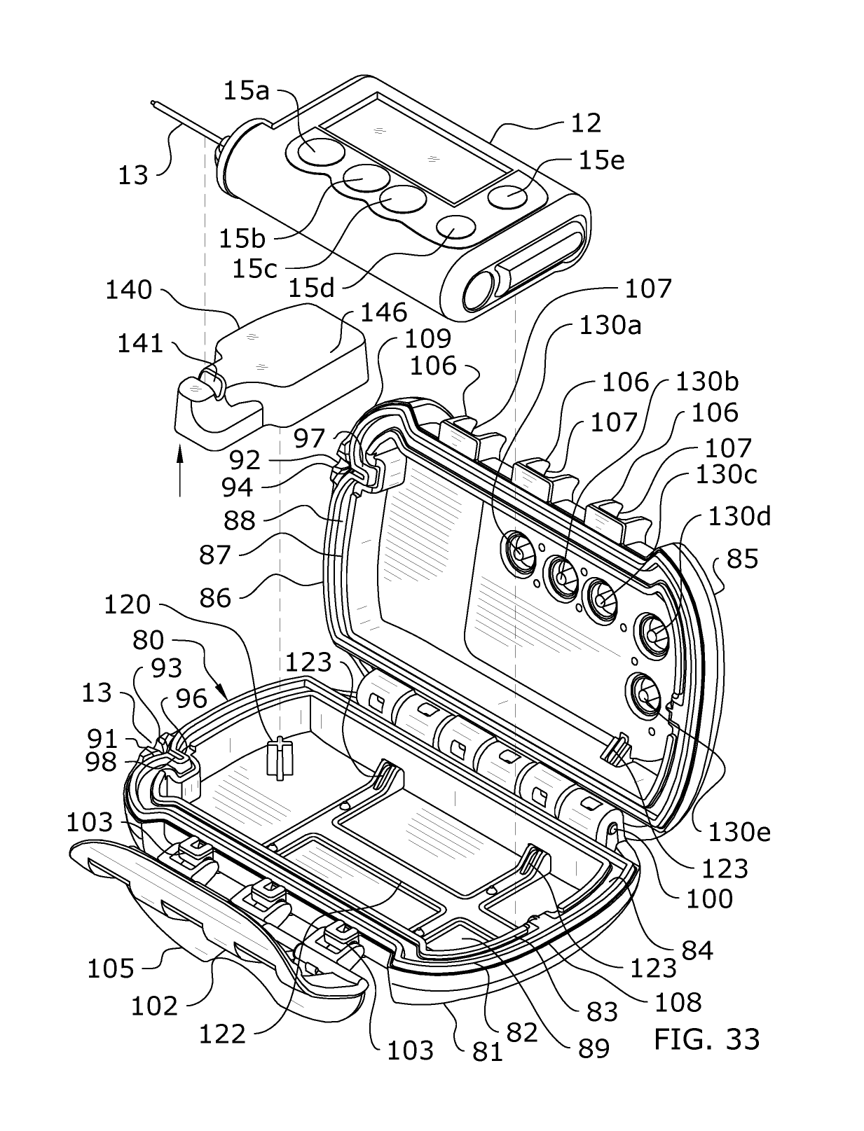

FIGS. 25-43 illustrate an example embodiment of a case 80 which shares a similar construction with the case 20 shown in FIGS. 22-24. It should be understood by one of ordinary skill in the art that the case 80 may be adapted to store and retain any number of different types of devices or objects, including a medical device 12 such as a pump or the like. The case 80 may be utilized with medical devices 12 which include a tube 13 that would benefit from being sealed, or it may be utilized with medical devices 12 which do not include a tube 13 extending therefrom.

The case 80 is illustrated as having a first portion 81 and a second portion 85 which close to form a compartment 89 adapted to sealably store and retain a medical device 12 therein. The case 80 generally has an opened state in which its compartment 89 is accessible to place a medical device 12 therein. In the opened state, the first and second portions 81, 85 of the case 80 are separated from each other.

The case 80 also has a closed state in which the first and second portions 81, 85 are closed against each other. When in a closed state, the compartment 89 is sealed so as to prevent escape of any fluids from the medical device 12 and to prevent ingress of fluids into compartment 89. The compartment 89 is preferably sealed whether a tube 13 extends out of the case 80 or not.

The first portion 81 and second portion 85 are preferably hingedly secured to each other via a case hinge 100 as shown in the figures, though it is appreciated that other configurations may be utilized to adjust the case 80 between its open state and its closed state. The present invention should not be construed as limited to hinged configurations. For example, one embodiment may include completely separable first and second portions 81, 85. A first seal 96 on the first portion 81 of the case 80 and a second seal 97 on the second portion 85 of the case 80 are operable to seal around a tube 13 extending from the medical device 12. When there is no tube 13 extending from the medical device 12 (and thus no tube 13 extending out of the case 80), the first and second seals 96, 97 will still seal against each other to seal the compartment 89 of the case 80 against fluid ingress.

The case 80 may be comprised of any variety of shapes, sizes and configurations to accommodate a wide range of medical devices 12. While the figures illustrate the case 80 as being generally rectangular in shape, it should be appreciated that the case 80 could be any number of shapes, including round, triangular, polygonal and the like. Larger cases 80 may be provided for larger medical devices 12 and smaller cases 80 may be provided for more compact medical devices 12.

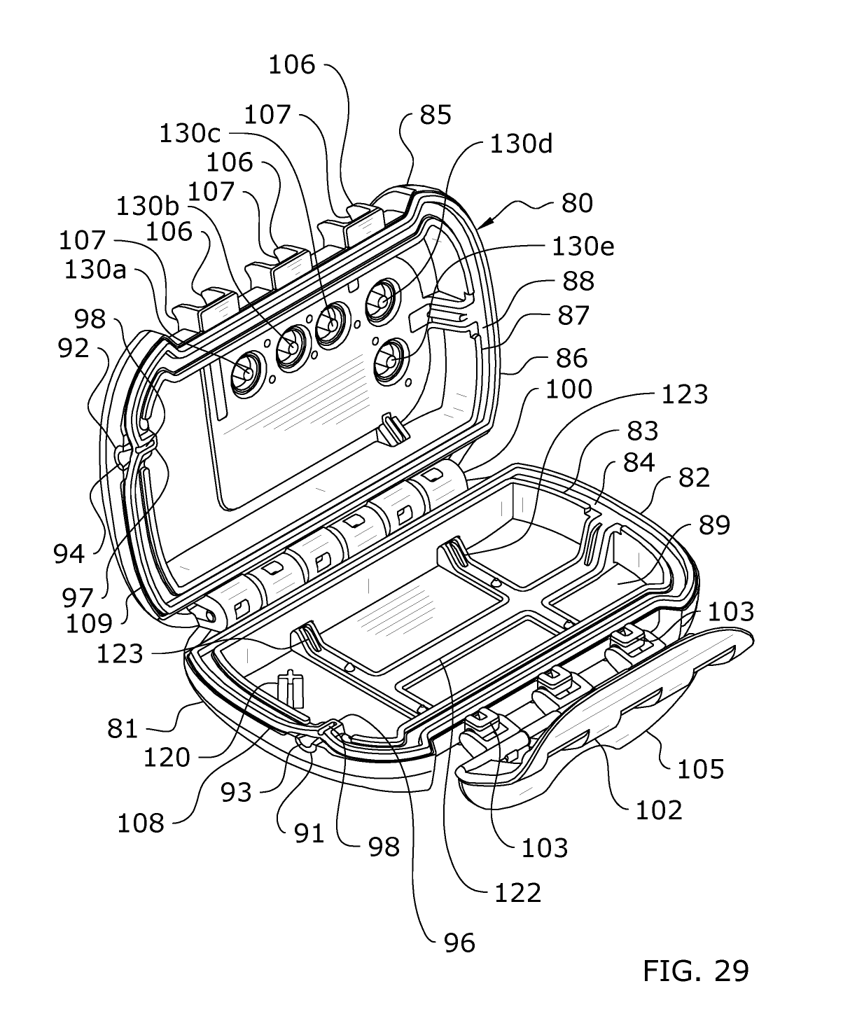

The case 80 will generally comprise a first outer edge 108 on the first portion 81 and a second outer edge 109 on the second portion 85. As best shown in FIGS. 29-36, the first portion 81 may comprise a first outer edge 108 which extends around the outer perimeter of the first portion 81. Similarly, the second portion 85 of the case 80 may comprise a second outer edge 109 which extends around the outer perimeter of the second portion 85. In some embodiments, the first outer edge 108 will be flush with the second outer edge 109 when the case 80 is in its closed state.

The first portion 81 of the case 80 may include a first perimeter seal 82 positioned at or near the first outer edge 108. Similarly, the second portion 85 of the case 80 may include a second perimeter seal 86 at or near the second outer edge 109. The outer perimeter seals 82, 86 may be offset with respect to the outer edges 108, 109 of the case 80. For example, the outer perimeter seals 82, 86 may be spaced away from the outer edges 108, 109 of the case 80 such that the outer edges 108, 109 of the case form flanges with respect to the outer perimeter seals 82, 86. When the case 80 is closed, the outer perimeter seals 82, 86 may seal against each other to prevent ingress of fluids into the compartment 89.

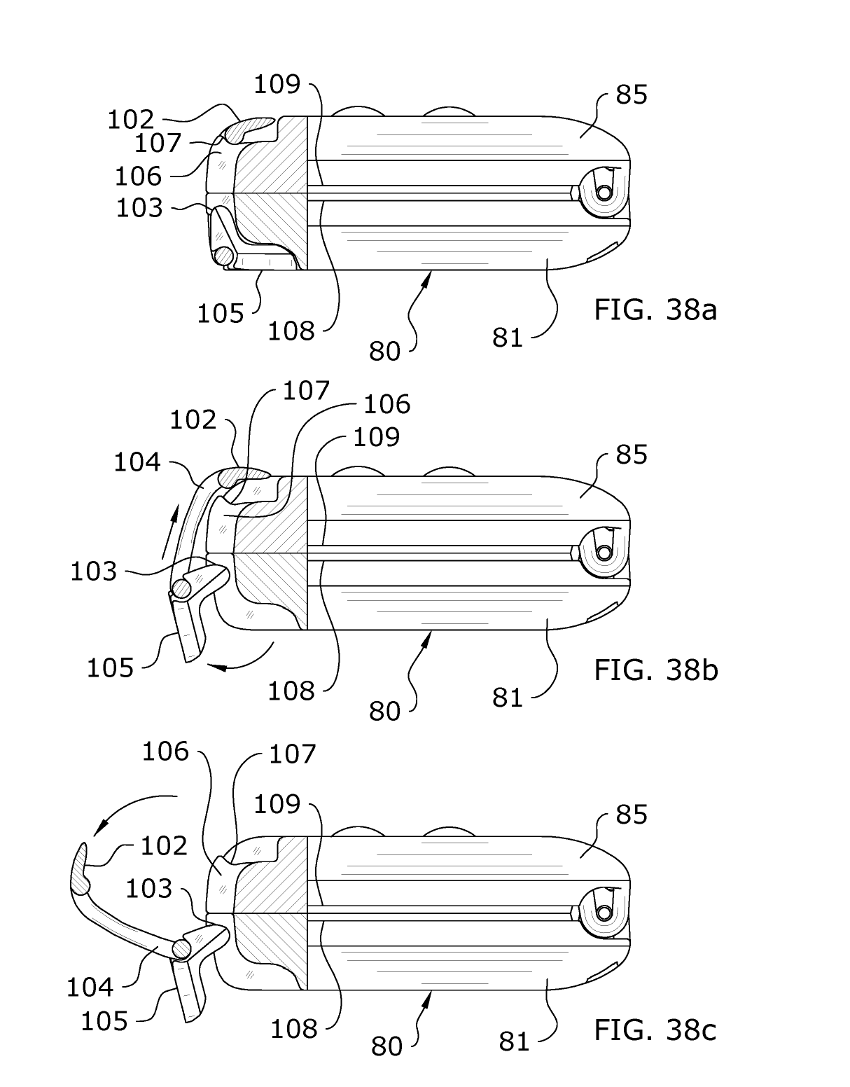

The perimeter seals 82, 86 may vary in construction, but will generally be comprised of a sealing material such as rubber or the like which extends around the perimeter of the first portion 81 and second portion 85 of the case 80. The first perimeter seal 82 is adapted to seal against the second perimeter seal 86 when the case 80 is in a closed state as shown in FIGS. 38-41. The perimeter seals 82, 86 may be transparent or partially transparent in some embodiments.

The perimeter seals 82, 86 will press against each other in a manner which fully seals the compartment 89 of the case 80 when the case 80 is closed by engaging the first portion 81 with the second portion 85. Thus, fluids will be prevented from entering the compartment 89 via ingress between the first and second portions 81, 85.

As shown in FIG. 29, the case 80 may include spacers 83, 87 which contact each other when the case 80 is in its closed position. The first portion 81 of the case 80 may include first spacers 83 and the second portion 85 of the case 80 may include second spacers 87 as shown in the figures. The first spacers 83 of the first portion 81 of the case 80 will contact and abut against the second spacers 87 of the second portion 85 of the case 80 when the case 80 is in its closed position so as to prevent overcompression of the seals 82, 86, 96, 97.

The spacers 83, 87 will generally comprise raised ribs or the like. The first spacers 83 may extend upwardly from the first portion 81 of the case 80 and the second spacers 87 may extend downwardly from the second portion 85 of the case 80 such that they abut against each other when the case 80 is closed. The shape, size, configuration, and orientation of the spacers 83, 87 may vary. In some embodiments, the spacers 83, 87 may be omitted entirely.

As best shown in FIGS. 40-42 and discussed above, the first outer edge 108 of the first portion 81 of the case 80 may overlap the first perimeter seal 82 to form a flange. Thus, the first perimeter seal 82 may be inwardly offset by a distance from the first outer edge 108 of the first portion 81 of the case 80. Similarly, the second portion 85 of the case 80 may overlap the second perimeter seal 86 to form a flange. Thus, the second perimeter seal 86 may be slightly inwardly offset by a distance from the second outer edge 109 of the second portion 85 of the case 80.

The distance of offset between the outer edges 108, 109 of the case 80 and the outer perimeter seals 82, 86 may vary in different embodiments. When the case 80 is in a closed state, a perimeter groove 98 may be formed which partially or fully surrounds the case 80 between the first and second portions 81, 85. The perimeter groove 98 is defined by the outer perimeter seals 82, 86 and the outer edges 108, 109 which extend outwardly with respect to the outer perimeter seals 82, 86. This perimeter groove 98 may aid in preventing ingress of fluids into the compartment 89 of the case 80 by creating a channel through which the fluids may pass out of and away from the case 80 rather than pooling up.

The case 80 is adapted to allow a tube 13 of a medical device 12 stored within the compartment 89 of the case 80 to exit the case 80 in a manner which seals the compartment 89 whether the tube 13 is present or absent. While the outer perimeter seals 82, 86 may provide this functionality, added sealing functionality may be provided by a tube seal assembly 90 comprising a first seal 96 and a second seal 97.

The case 80 may include tube guides 91, 92 which guide any tube 13 exiting the case 80. The first portion 81 of the case 80 may include a first tube guide 91 and the second portion 86 of the case 80 may include a second tube guide 92. The first tube guide 91 comprises a first channel 93 and the second tube guide 92 comprises a second channel 94 such that, when the case 80 is in its closed state, the first and second channels 93, 94 combine to form a passage 95 through which the tube 13 may exit the case 80. This passage 95 is preferably sealed when there is no tube 13 in the case 80 and the case 80 is in its closed state.

The tube seal assembly 90, comprising a first seal 96 on the first portion 81 of the case 80 and a second seal 97 on the second portion 85 of the case 80, is best shown in FIGS. 29-36. The first seal 96 is generally positioned on the first portion 81 of the case 80 aligned with the first tube guide 91. The second seal 97 is generally positioned on the second portion 86 of the case 80 aligned with the second tube guide 92. In the exemplary embodiment of the figures, the first seal 96 is positioned behind the first tube guide 91 and the second seal 97 is positioned behind the second tube guide 92.

The first seal 96 may comprise a U-shaped seal which extends into the compartment 89 of the case 80 from the passage 95. The second seal 97 may comprise a U-shaped seal which extends into the compartment 89 of the case 80 from the passage 95 such that, when the case 80 is closed, the first and second seals 97, 98 seal against each other when no tube 13 is present. If a tube is present, the first and second seals 97, 98 deformed around the tube 13 to seal off the passage 95 surrounding the tube 13. The first and second seals 96, 97 may be integrally formed with the outer perimeter seals 82, 86 or may be discrete, separate structures which intersect the outer perimeter seals 82, 86.

As best shown in FIGS. 29-36, the medical device case 80 may include a case hinge 100 which hingedly connects the first portion 81 of the case 80 to the second portion 85 of the case 80. Various types of case hinges 100 may be utilized, and thus the exemplary figures and descriptions herein should not be construed as limiting on the scope of hinge 100 available for use with the case 80.

As best shown in FIGS. 38a-c and 39a-c, the case 80 may include a clasp 101 which is utilized to lock the case 80 in its closed position to ensure that the case 80 is not unintentionally opened and to perfect the seal between the first portion 81 and second portion 85 of the case. Various types of clasps 101 may be utilized, and the following description should not be construed as limiting on the scope of the invention.

In the exemplary figures, an exemplary embodiment of a clasp 101 is shown which comprises a latch hook 102 and a latch lever 105. Both the latch hook 102 and the latch lever 105 are hingedly connected to the first portion 81 of the case 80 by a clasp hinge 103. The clasp hinge 103 is connected across the first portion 81 of the case 80; with the latch hook 102 extending upwardly and the latch lever 105 extending downwardly such as shown in FIG. 38c.

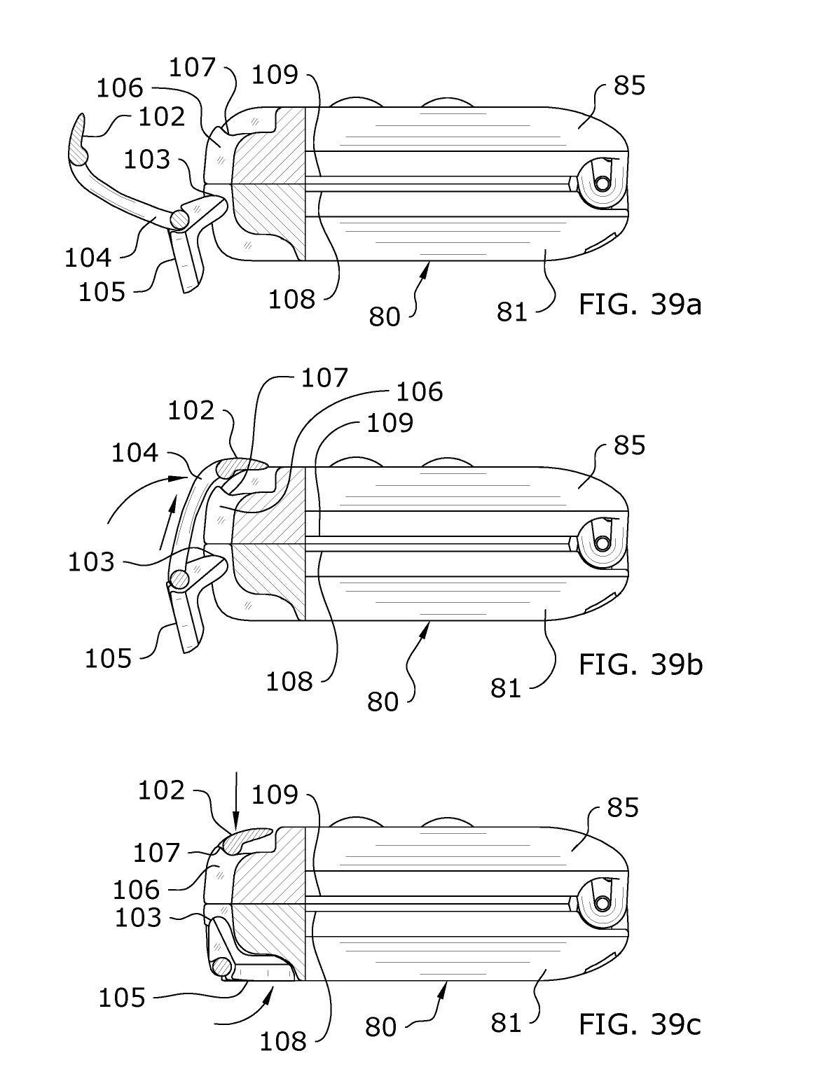

The latch hook 102 is hingedly connected to swing between approximately 90 degrees and 180 degrees with respect to the case 80. The latch hook 102 swings upwardly to engage with the second portion 85 of the case 80 as shown in FIG. 39b and downwardly to disengage from the second portion 85 of the case 80 as shown in FIG. 38c. The latch lever 105 is hingedly connected to swing between approximately 90 degrees and 0 degrees with respect to the case 80. The latch lever 105 swings downwardly to engage the clasp 101 as shown in FIG. 39c or upwardly to disengage the clasp 101 as shown in FIG. 38b.

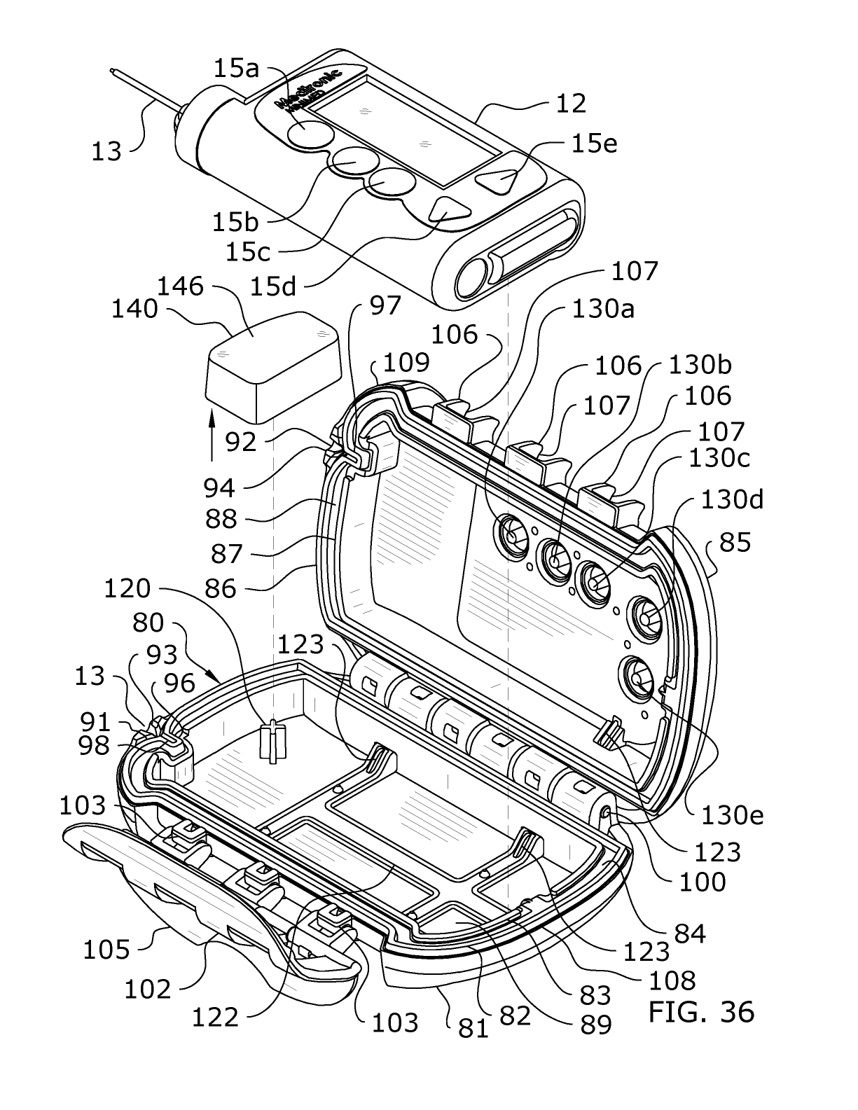

The latch hook 102 comprises a member which swings upwardly to engage with the second portion 85 of the case 80. As shown in the figures, the latch hook 102 may include one or more latch openings 104 which are adapted to receive and engage with corresponding clasp anchors 106 to lock the case 80 in the closed state. As shown in FIG. 36 and elsewhere, one or more of the clasp anchors 106 extend outwardly from the second portion 85 of the case 80. When the case 80 is closed and the latch hook 102 is swung upwardly, the clasp anchors 106 will insert into the latch openings 104 and removably engage therewith to lock the case 80 closed. The clasp anchors 106 may each include tapered portions 107 as best shown in FIGS. 29-36 which retain the clasp 102 in a locked position absent sufficient force to pull the clasp openings 104 over the tapered portions 107 to be release the clasp 100.

The latch lever 105 is connected to the latch hook 102 to aid in engaging or disengaging the latch hook 102 with the second portion 85 of the case 80. The latch lever 105 may be pulled downwardly to engage the latch hook 102 as shown in FIG. 39c or may be pulled upwardly to disengage the latch hook 102 as shown in FIG. 38b.

Due to the varying size of different medical devices 12, some example embodiments may include a spacer block 140 which may be removably installed into the compartment 89 of the case 80 to accommodate differently-sized medical devices 12.

For example, if the compartment 89 is substantially larger in area than the medical device 12, a spacer block 140 may be removably positioned within the compartment 89 to reduce the effective width of the compartment 89 such that the medical device 12 does not bounce around in the compartment 89. Preferably, the medical device 12 will be snugly fit within the compartment 89 such that movement of the medical device 12 within the compartment 89, which can lead to damage or leakage, is minimized or prevented outright.

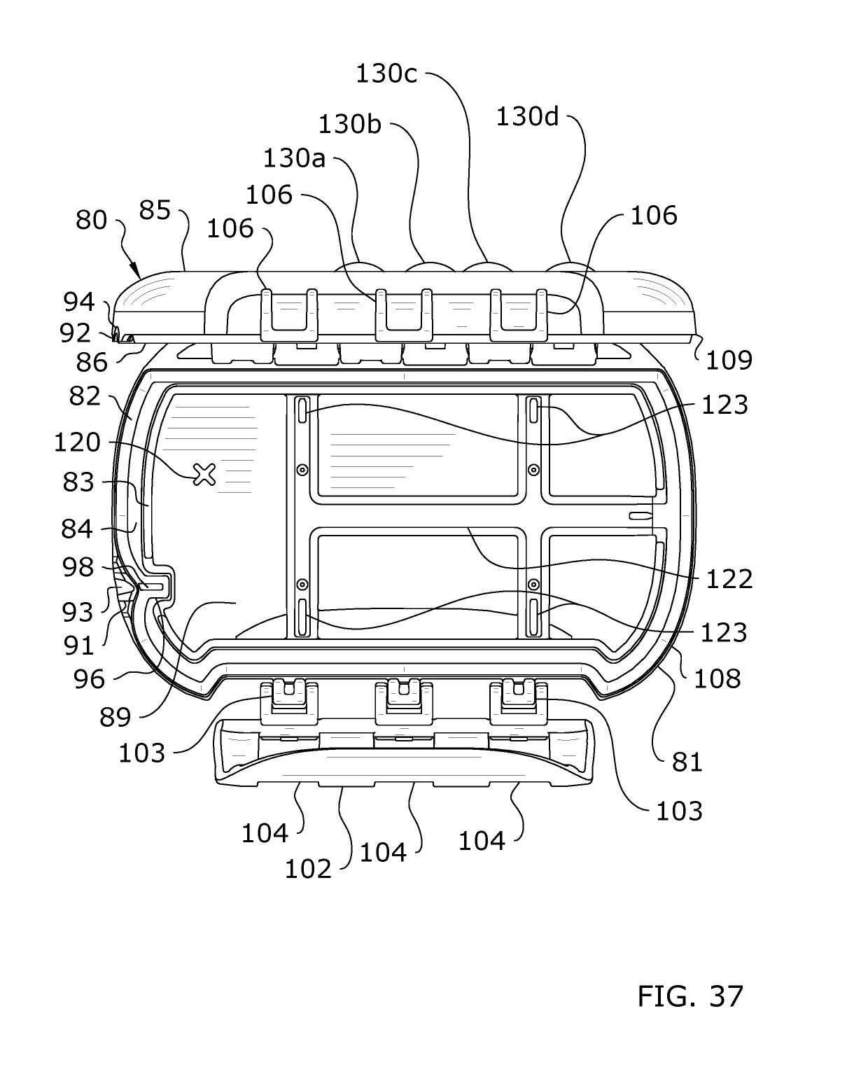

In addition or as an alternative to the use of a spacer block 140, the case 80 may include internal supports 122 and edge supports 123 within the compartment 89 as best shown in FIG. 37. The internal supports 122 may comprise ribbing along the compartment 89 which comprises a non-slide material. The edge supports 123 may comprise triangular supports along the inner edges of the compartment 89 to support the medical device 12 such as shown in FIG. 36.

The spacer block 140 may be removably connected to the case 80 in various manners, such as by clasps, adhesives, fasteners such as hook-and-loop fasteners, brackets, bolts, and the like. In the exemplary embodiment shown in the figures, the case 80 may include a block mount 120 which is positioned in the compartment 89 to engage with a spacer block 140. The block mount 120 may comprise a projection which extends into the compartment 89 and is adapted to be inserted to engage with a corresponding receiver 142 on the spacer block 140. In the embodiment shown in the figures, the block mount 120 may comprise an X-shaped cross-section to prevent rotation of the spacer block 140 when installed thereto.

Various types of spacer blocks 140 may be utilized with different embodiments of the case 80 or to accommodate different types of medical devices 12. Thus, the exemplary spacer blocks 140 illustrated in the figures should not be construed as limited by the exemplary shape, size, configuration, and/or orientation shown. The embodiments shown in the figures are merely for exemplary purposes, as the overall shape, size, configuration, and/or orientation of the spacer block 140 may vary widely in different embodiments or to suit different applications. In some embodiments, multiple spacer blocks 140 may be supported.

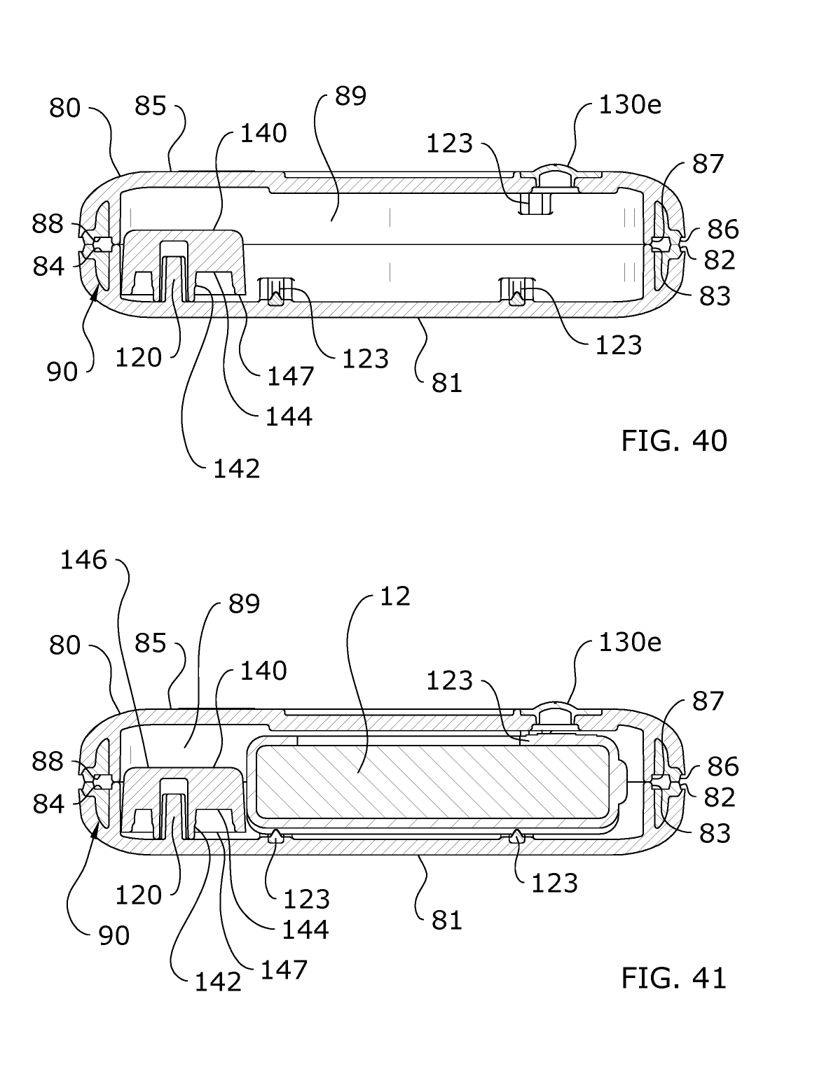

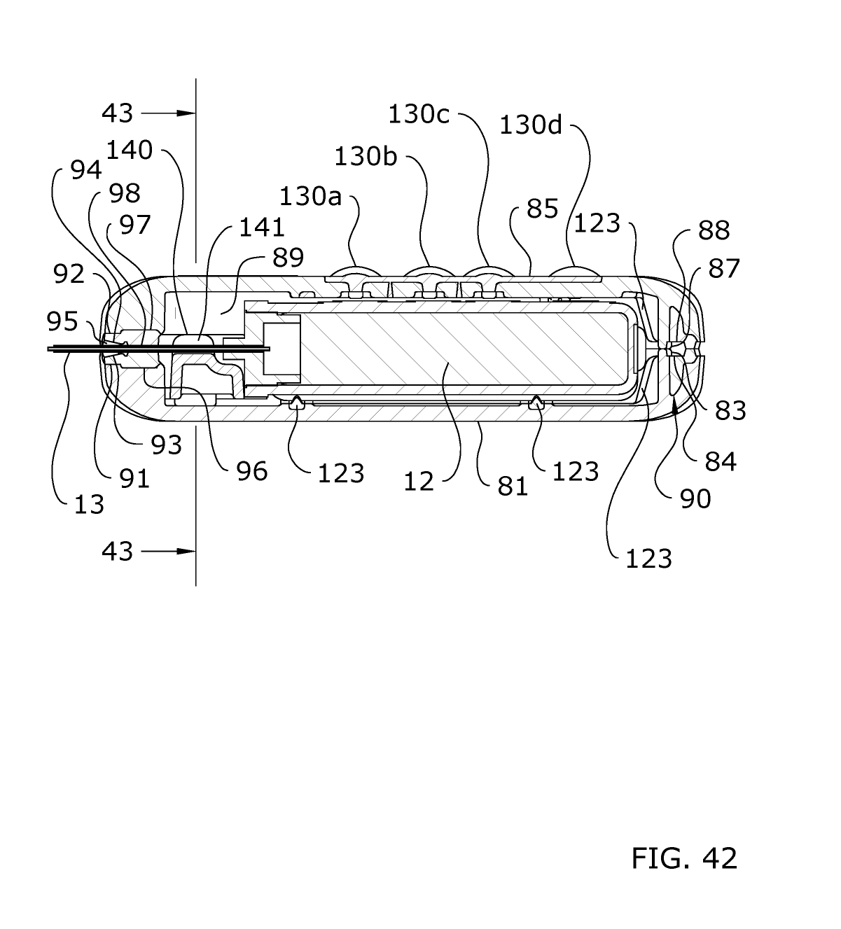

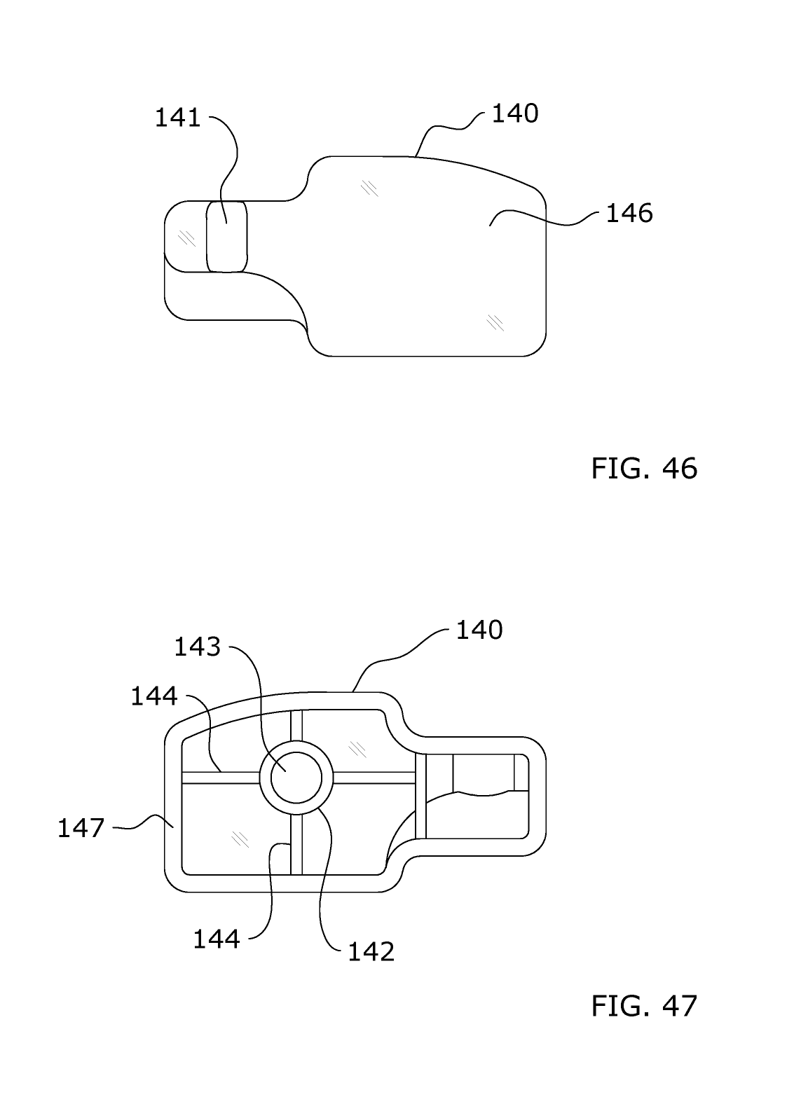

FIGS. 42-49 illustrate exemplary spacer blocks 140. As shown, the spacer block 140 will generally comprise an upper end 146 and a lower end 147. The lower end 147 of the spacer block 140 will generally include a receiver 142 which is adapted to removably engage with the corresponding block mount 120 in the compartment 89 of the case 80. The receiver 142 may comprise an opening 143 having various cross-sections to releasably engage with the block mount 120. The block mount 120 is inserted into the opening 143 of the receiver 142 to releasably engage the spacer block 140 with the block mount 120. Ribs 144 may be provided which reinforce the receiver 142 as shown in FIGS. 47 and 51.

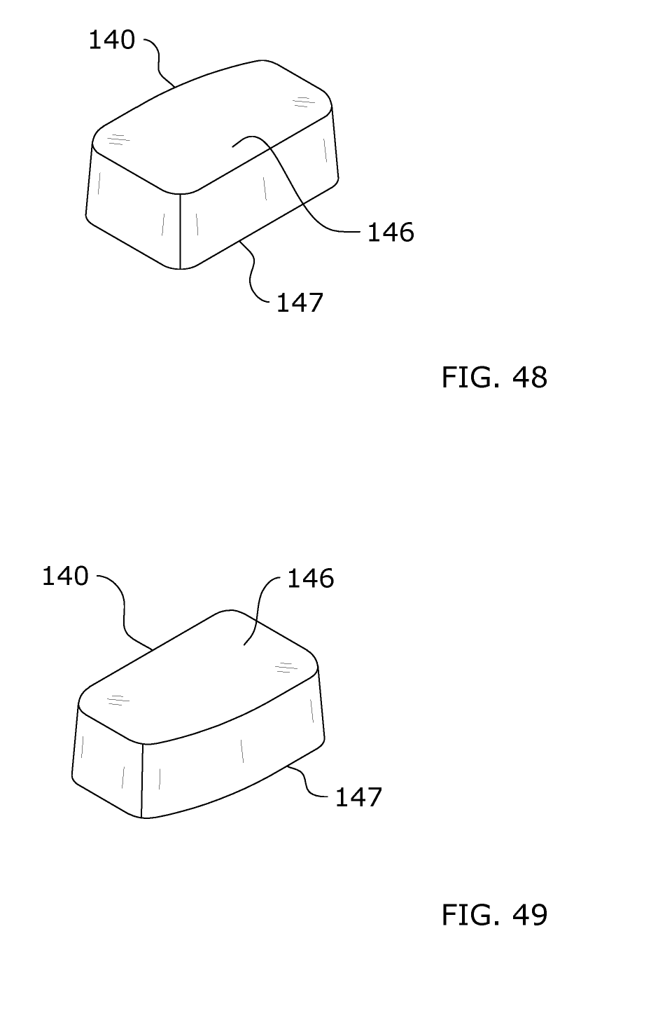

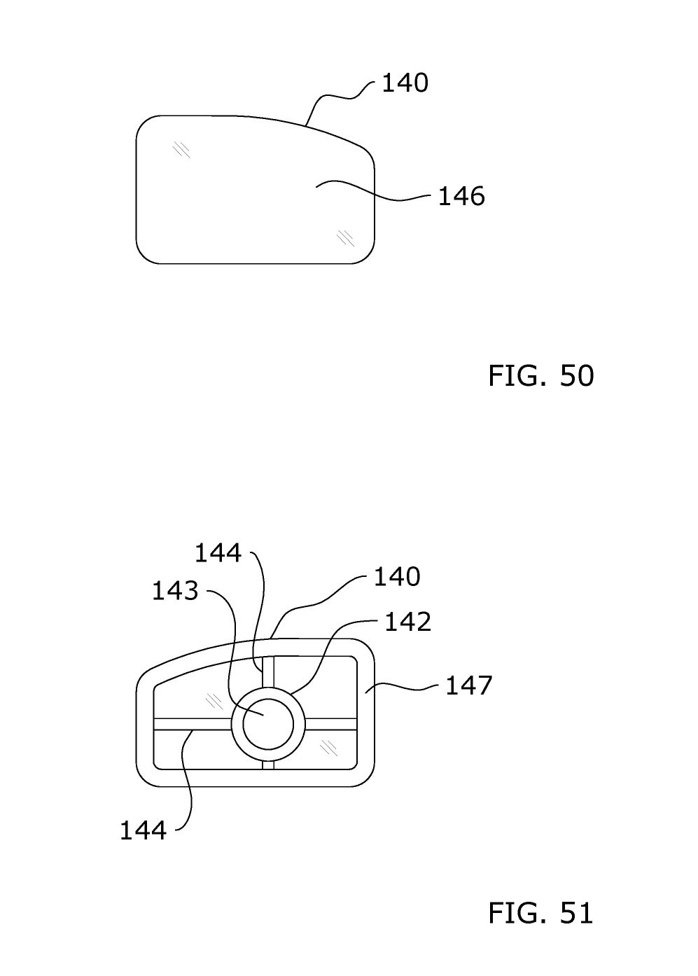

Depending on the shape and size of the spacer block 140, the spacer block 140 may include a block channel 141 through which the tube 13 may pass as it exits the case 80. Such a block channel 141 is unnecessary with respect to spacer blocks 140 which do not pass in front of the medical device 12. FIGS. 48-51 illustrate such a spacer block 140 without a block channel 141.

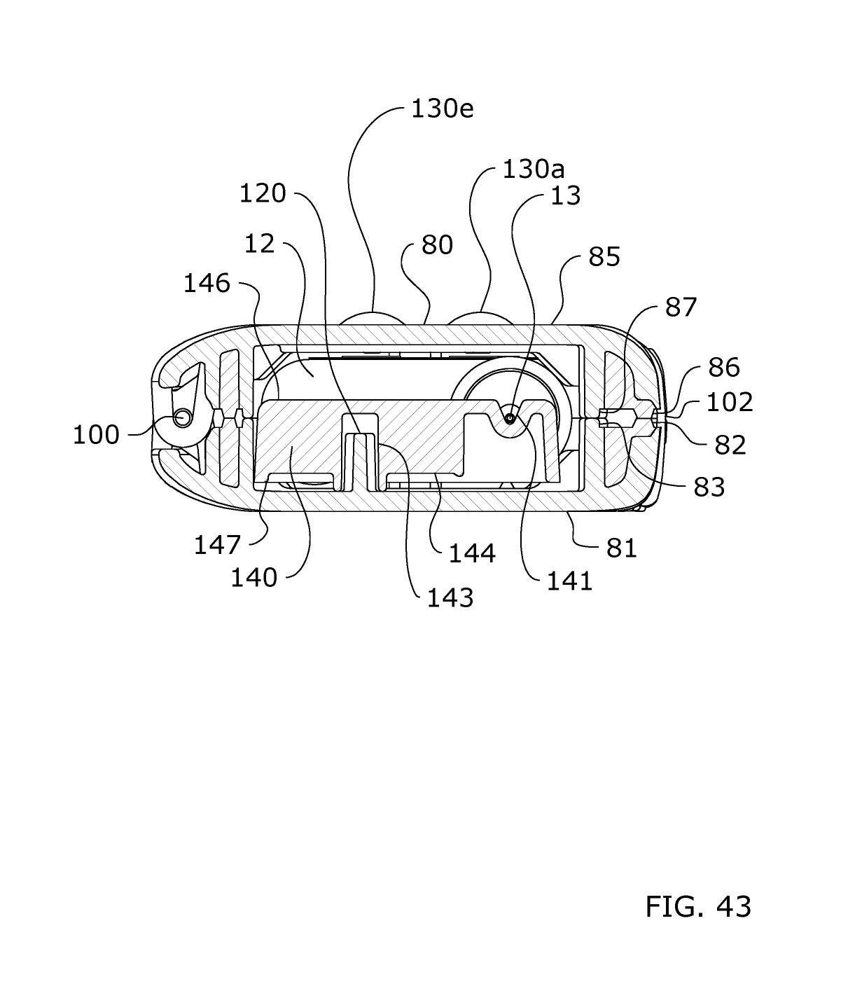

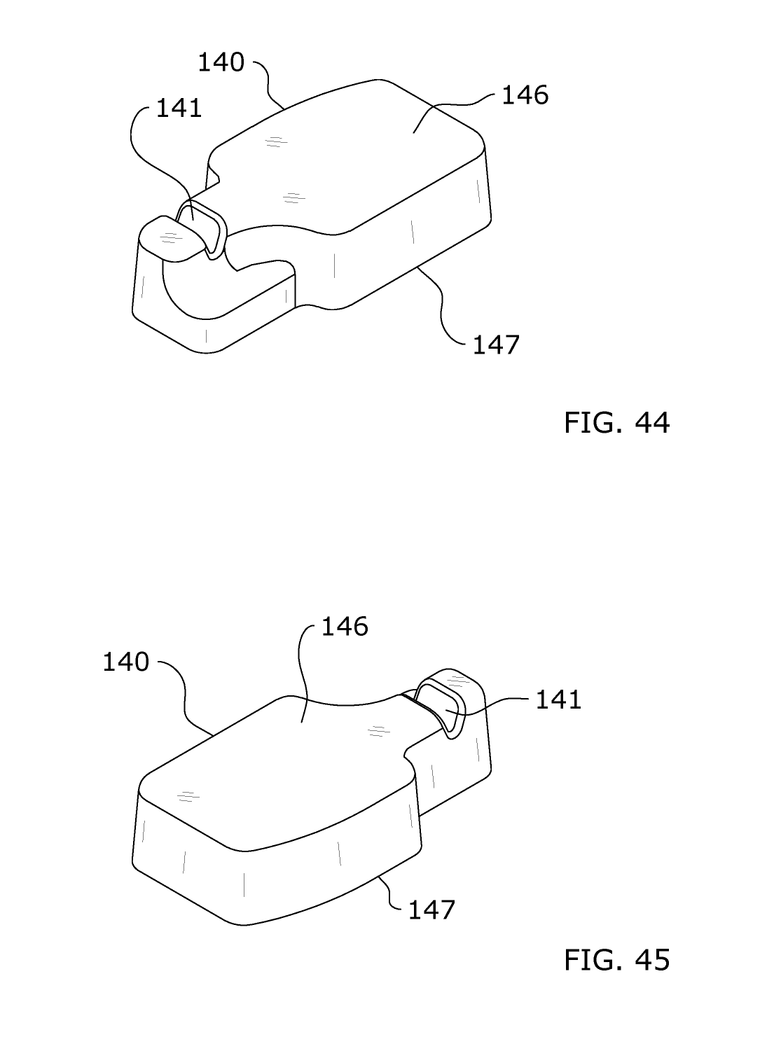

FIGS. 44-47 illustrate a spacer block 140 with a block channel 141. As best shown in FIGS. 42 and 43, the block channel 141 is in alignment with the passage 95 of the tube guides 91, 92 such that the tube extends through the block channel 141 and out of the case 80 through the tube guides 91, 92. The seals 96, 97 will seal around the tube 13 in such embodiments as discussed above.

Some embodiments of the case 80 may include pass-through controls 130a-e to allow control of the device controls 15a-e of the medical device 12. For example, a first pass-through control 130a of the case 80 will be positioned directly above a corresponding device control 15a such that pressing down on the control 130a on the case 80 will cause the underlying device control 15a to be depressed. The number, orientation, and positioning of the controls 130a-e may vary in different embodiments and should not be construed as limited by the exemplary figures.

In use, the case 80 is first opened by releasing the clasp 101 using the latch lever 105 to such that the latch hook 102 disengages from the clasp anchors 106 as shown in FIGS. 38a-c. With the clasp 101 released, the case 80 may be hingedly opened by grasping the second portion 85 of the case 80 and pulling it upwardly and away from the first portion 81 of the case 80 to expose the compartment 89 as shown in FIG. 29.

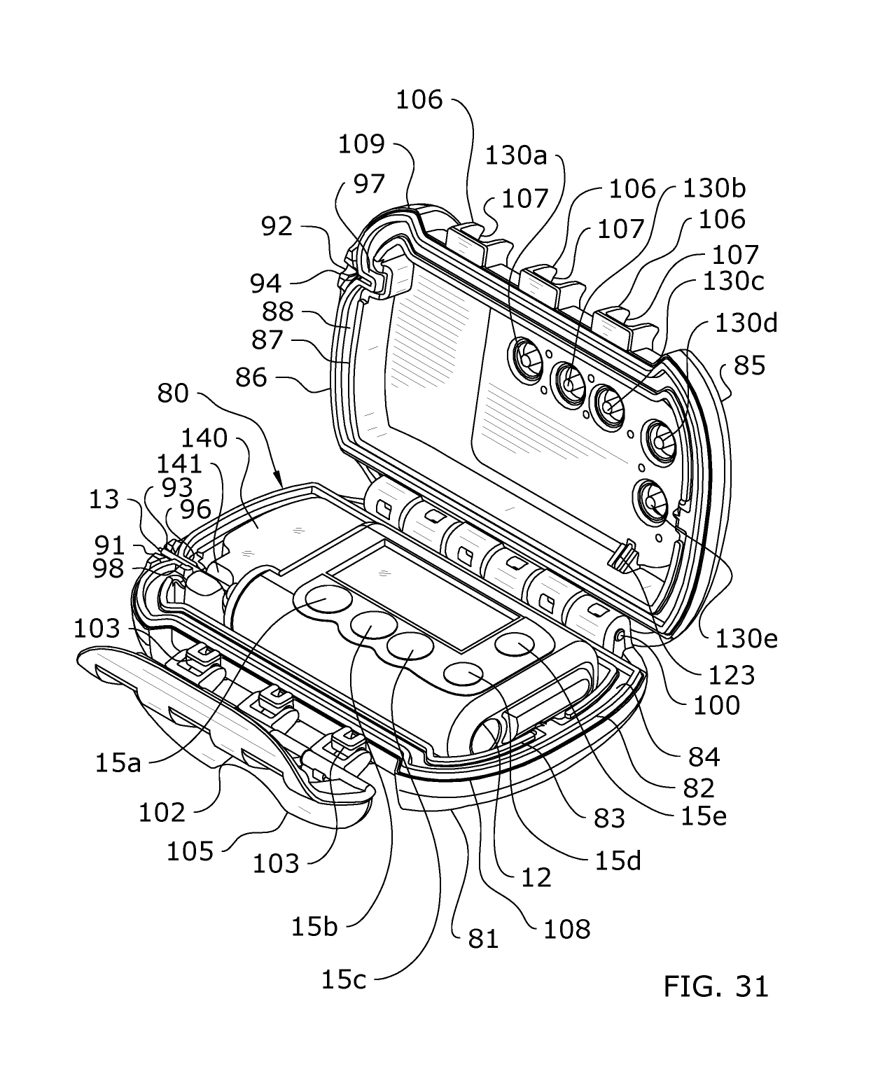

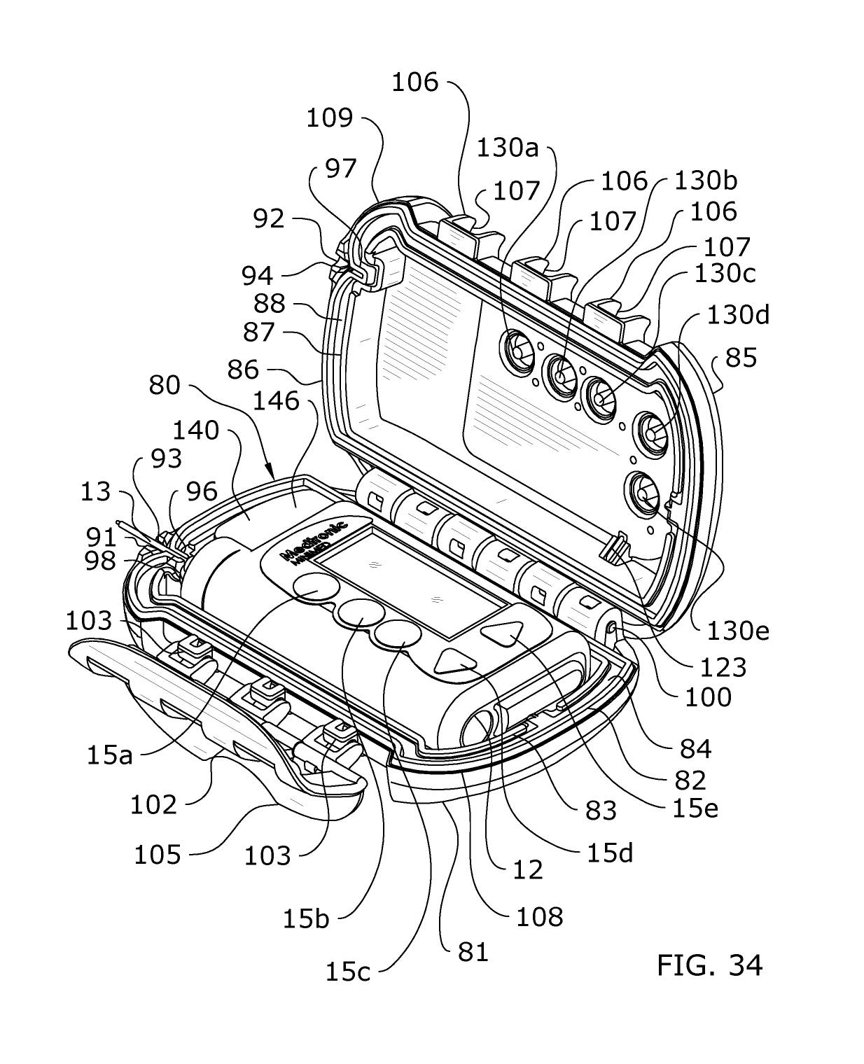

A medical device 12 may then be placed in the compartment 89 of the case 80 as shown in FIGS. 31 and 34. The medical device 12 should preferably be positioned within the first portion 81 of the case 80 to snugly fit between the edge supports 123 and rest upon the internal supports 122 of the case 80.

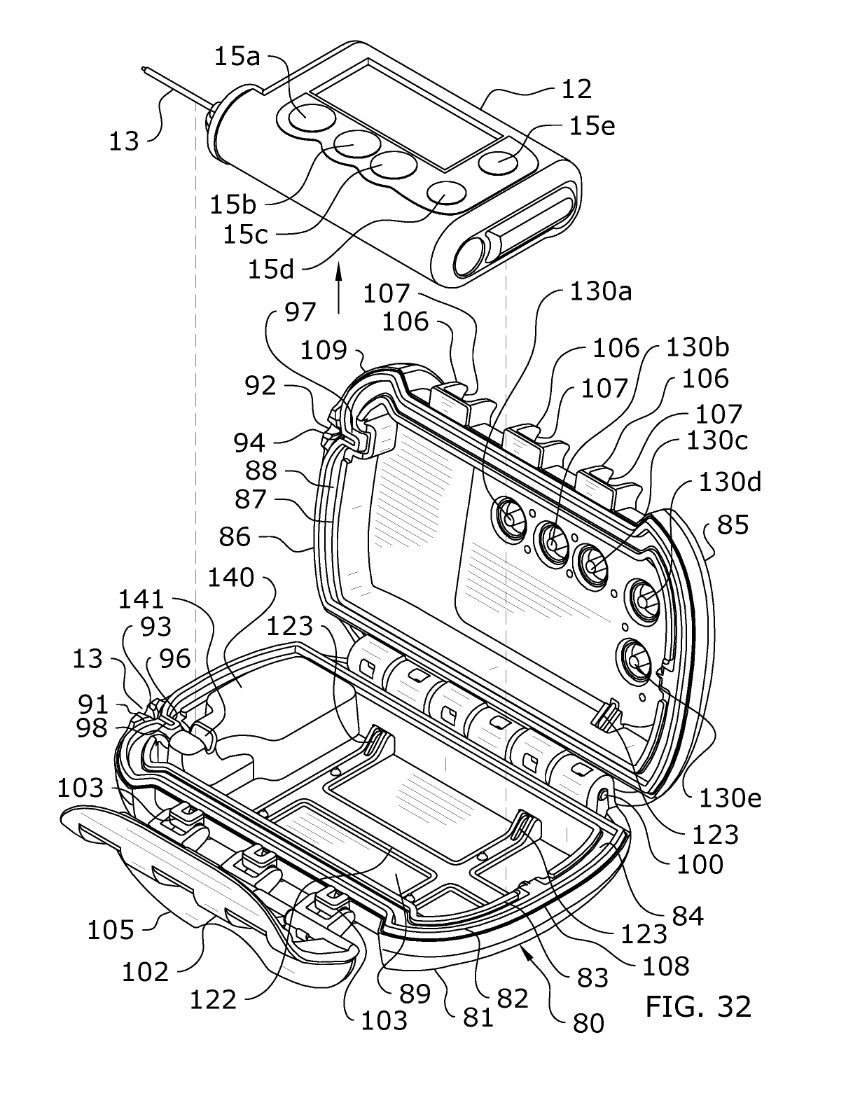

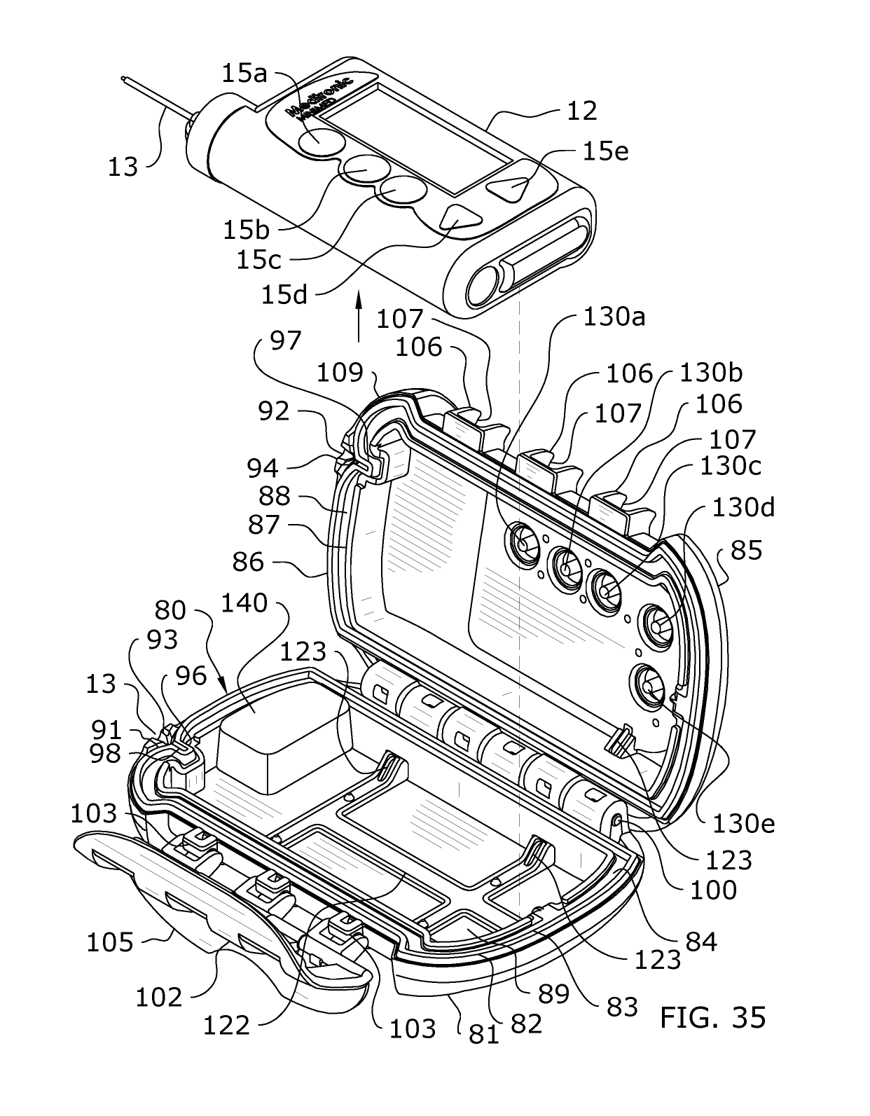

If the medical device 12 is smaller than the compartment 89, a spacer block 140 may be utilized. The spacer block 140 is first installed onto the block mount 120 within the compartment 89 as shown in FIGS. 32 and 35. The medical device 12 may then be positioned within the case 80 to rest up against the spacer block 140 so as to prevent movement of the medical device 12 within the case 80 as shown in FIG. 39.

With the medical device 12 (and any optional spacer blocks 140) positioned within the case 80, the case 80 may be closed. The second portion 85 of the case 80 is grasped and pulled downwardly to contact the first portion 81 of the case 80. If a tube 13 is present, the seals 96, 97 and/or perimeter seals 82, 86 will seal around the tube 13 as shown in FIGS. 40 and 41. The spacers 83, 87 will prevent overcompression of the seals 82, 86, 96, 97.

If no tube is present, the seals 96, 97 and/or perimeter seals 82, 86 will seal against each other to seal off the passage 95 through which the tube 13 would have extended as shown in FIGS. 40 and 41. Thus, the compartment 89 is sealed from ingress of fluids whether a tube 13 is present or not. Any fluids external of the case 80 will be prevented from ingress by the perimeter groove 98, which will cause the fluids to be routed out and away from the case 80.

With the case 80 closed, it may be locked by engaging the clasp 101. The latch hook 102 may be grasped and swung upwardly such that the clasp anchors 106 on the second portion 85 of the case 80 engage with the latch openings 104 of the latch hook 102 as shown in FIGS. 39a and 39b. The latch lever 105 may then be pulled downwardly to lock the engagement as shown in FIG. 39c. When so engaged, the case 80 is locked in its closed state awaiting future use as shown in FIGS. 25-27.

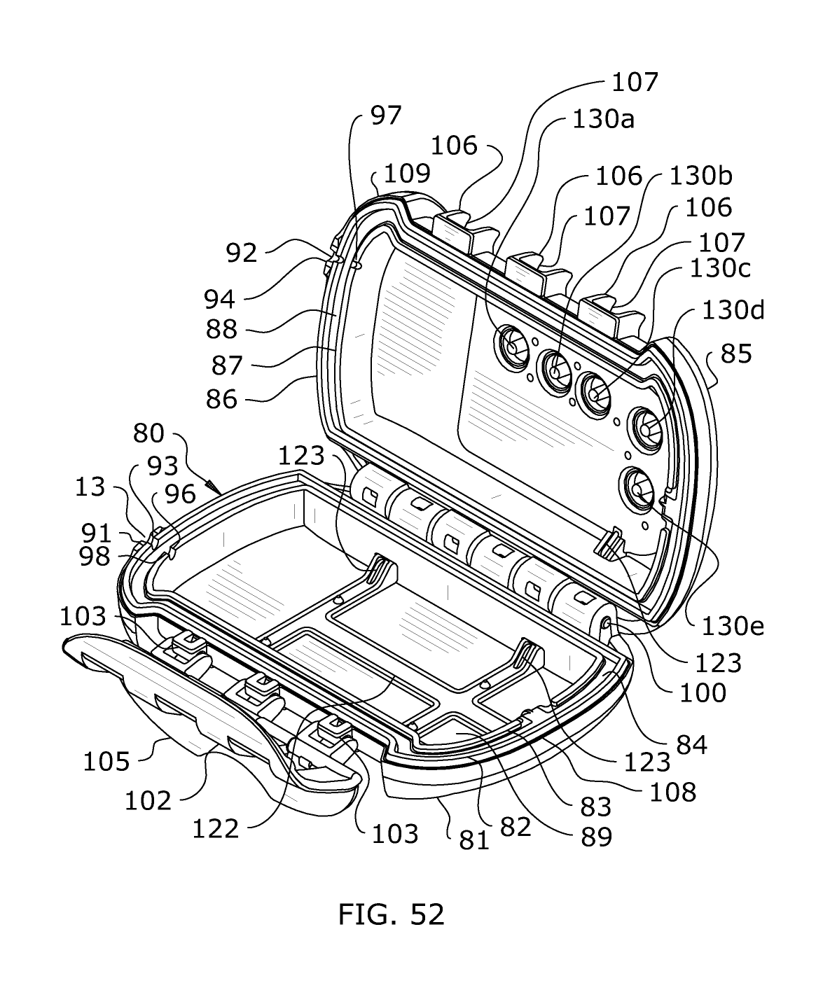

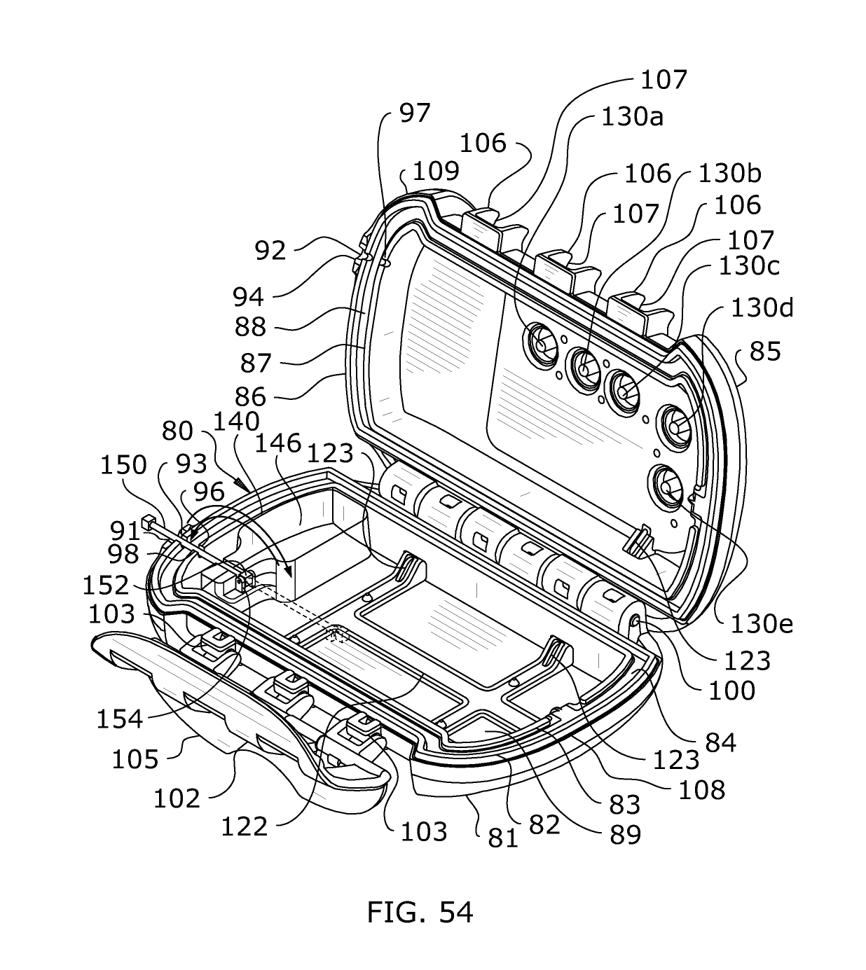

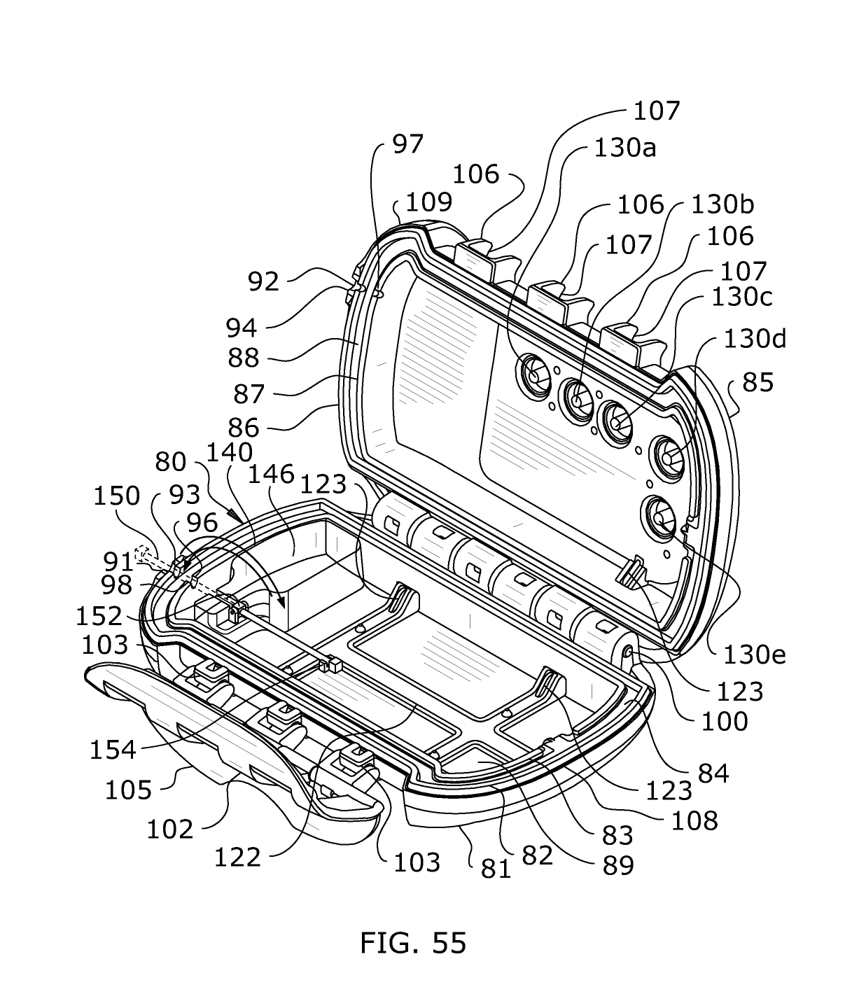

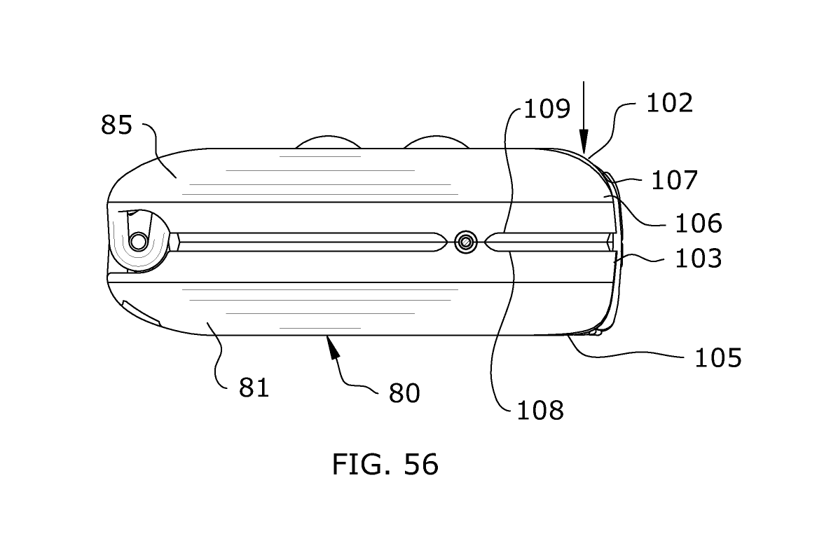

FIGS. 52-56 illustrate an alternate embodiment of the medical device case 80 in which the first and second seals 96, 97 surrounding the passage 95 are omitted and a plug 150 is instead utilized to seal the passage 95 when no tube 13 is present. In such an embodiment, the medical device case 80 still includes the perimeter seals 82, 86 which terminate at the passage 95 such that the perimeter portions of the medical device case 80 are sealed when the medical device case 80 is in a closed position.

As shown in FIG. 52, such a case will generally include a passage 95 adapted to seal around the tube 13 when it is present, but does not include the first and second seals 96, 97. Such an embodiment will still seal the medical device case 80 when it is closed around a tube 13 exiting through the passage 95; with the passage 95 itself providing the sealing around the tube 13.

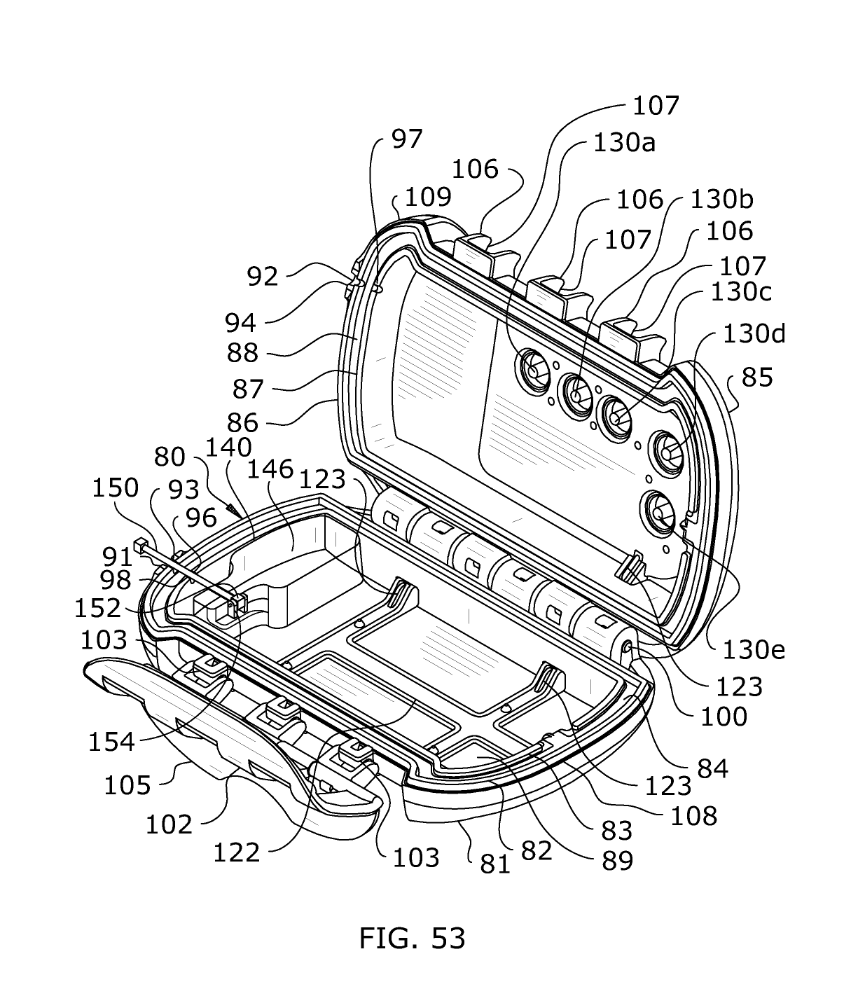

When a tube 13 is not present in the passage 95 and the medical device case 80 is closed, a plug 150 may be inserted within the passage 95 to seal the passage 95 absent the tube 13 instead of the first and second seals 96, 97. The plug 150 may be utilized with the spacer block 140 installed such as shown in FIGS. 53-55; with the plug 150 being positioned such that it extends through both the block channel 141 of the spacer block 140 and the passage 95 of the closed medical device case 80.

Additionally, the plug 150 may be utilized when no spacer block 140 is installed by simple plugging the passage 95. In either case, the combination of the plug 150 sealing the passage 95 and the perimeter seals 82, 86 sealing the perimeter will ensure a complete seal of the compartment 89 of the medical device case 80 even when no tube 13 is present in embodiments omitting the first and second seals 96, 97.

The shape, size, orientation, and configuration of the plug 150 may vary in different embodiments. Thus, the shape, size, orientation, and configuration of the plug 150 shown in the exemplary figures should not be construed as limiting on the scope of the invention. In the exemplary figures, the plug 150 is illustrated as comprising an elongated, cylindrical member which may include a grasping portion such as the cube-shaped extension illustrated on the distal end of the plug 150. Such an extension may make it easier to grasp the plug 150 to put it in or out of position to seal the passage 95.

The plug 150 may be fully removable from the case 80 such that it may be stored elsewhere. In such an embodiment, when not in use, the plug 150 may be attached to another portion of the medical device case 80 (either inside or outside) or may be carried elsewhere such as in a pocket. When desired, the plug 150 may be removably inserted within the passage 95 to seal the medical device case 80 along with the perimeter seals 82, 85 when no tube 13 is present. The plug 150 may be removed from the passage 95 and stored elsewhere when a tube 13 is present.

In some embodiments, the plug 150 may be secured to the medical device case 80 such as shown in FIGS. 53-55. In such embodiments, the plug 150 may be rotatably connected to the medical device case 80 such as by a hinge 152. A corresponding bracket 154 may be provided into which the plug 150 may be removable connected when not in use. When in use, the plug 150 may be rotated or pivoted into a second position in which it blocks the passage 95.

Unless otherwise defined, all technical and scientific terms used herein have the same meaning as commonly understood by one of ordinary skill in the art to which this invention belongs. Although methods and materials similar to or equivalent to those described herein can be used in the practice or testing of the present invention, suitable methods and materials are described above. All publications, patent applications, patents, and other references mentioned herein are incorporated by reference in their entirety to the extent allowed by applicable law and regulations. The present invention may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

What is claimed is:

1. A medical device case system, comprising:
a case for storing a medical device, wherein the case comprises a first portion and a second portion, wherein the first portion comprises a first outer edge and the second portion comprises a second outer edge, wherein the case has an opened state and a closed state;
a first seal on the first portion of the case; and
a second seal on the second portion of the case, wherein the first seal and the second seal are operable to seal around a tube extending from the medical device when the case is in the closed state.

2. The medical device case system of claim 1, wherein the first portion of the case comprises a first perimeter seal and the second portion of the case comprises a second perimeter seal.

3. The medical device case system of claim 2, wherein the first perimeter seal is offset with respect to the first outer edge of the first portion and wherein the second perimeter seal is offset with respect to the second outer edge of the second portion.

4. The medical device case system of claim 3, wherein the first outer edge of the first portion extends outwardly with respect to the first perimeter seal and wherein the second outer edge of the second portion extends outwardly with respect to the second perimeter seal.

5. The medical device case system of claim 3, further comprising a perimeter groove extending around the case when the case is in the closed state, wherein the perimeter groove is defined by the first outer edge of the first portion, the second outer edge of the second portion, the first perimeter seal, and the second perimeter seal.

6. The medical device case system of claim 2, wherein the first portion of the case comprises a first spacer and the second portion of the case comprises a second spacer.

7. The medical device case system of claim 1, wherein the first portion of the case comprises a clasp and wherein the second portion of the case comprises a clasp anchor, wherein the clasp is removably engaged with the clasp anchor to releasably lock the case in the closed state.

8. The medical device case system of claim 7, wherein the clasp is hingedly connected to the first portion of the case.

9. The medical device case system of claim 8, wherein the clasp comprises an opening adapted to receive and engage with the clasp anchor.

10. The medical device case system of claim 9, wherein the clasp anchor comprises a tapered portion for removably engaging the clasp with the clasp anchor.

11. The medical device case system of claim 7, wherein the clasp comprises a latch lever and a latch hook.

12. The medical device case system of claim 11, wherein the latch lever and the latch hook are each hingedly connected to the first portion of the case by a clasp hinge.

13. The medical device case system of claim 12, wherein the latch hook is adapted to engage with the second portion of the case.

14. The medical device case system of claim 13, wherein the latch lever is adapted to lock the latch hook in engagement with the second portion of the case.

15. The medical device case system of claim 1, further comprising a spacer block removably connected to the case.

16. The medical device case system of claim 15, wherein the first portion of the case comprises a block mount for removably engaging with the spacer block.

17. The medical device case system of claim 16, wherein the spacer block includes an opening, wherein the block mount is adapted to be removably inserted into the opening.

18. The medical device case system of claim 17, wherein the block mount comprises an X-shaped cross-section.

19. The medical device case system of claim 1, wherein the first portion of the case comprises a first perimeter seal, wherein the second portion of the case comprises a second perimeter seal, wherein the first seal is integral with the first perimeter seal and wherein the second seal is integral with the second perimeter seal.

20. The medical device case system of claim 1, wherein the first portion of the case comprises a first tube guide, wherein the second portion of the case comprises a second tube guide, wherein the tube extending from the medical device is adapted to extend through the first tube guide and the second tube guide.
